(19) United States
(12) Patent Application Publication
(10) Pub. No.: US 2026/0272276 A1
(43) Pub. Date: Sep. 17, 2026

(54) INFORMATION PROCESSING APPARATUS, INFORMATION PROCESSING METHOD, AND PROGRAM STORAGE MEDIUM STORING PROGRAM

(71) Applicant: Keyence Corporation, Osaka (JP)

(72) Inventors: Seima TABUSHI, Osaka (JP); Koki KARIYA, Osaka (JP)

(73) Assignee: Keyence Corporation, Osaka (JP)

(21) Appl. No.: 19/531,750

(22) Filed: Feb. 6, 2026

(30) Foreign Application Priority Data
Mar. 13, 2025 (JP) 2025-039961

(51) Int. Cl. A61B 1/04

(52) U.S. Cl. CPC A61B 1/043

(57) ABSTRACT

An information processing apparatus includes: a storage section configured to store a plurality of single fluorescence images acquired by different fluorescence filters, each of the single fluorescence images including metadata, and store a reference file that bundles some single fluorescence images from among the plurality of single fluorescence images; a search setting section configured to set fluorescence filter information as a search condition; a search section configured to search for the single fluorescence images and the reference file that satisfy the search condition on the basis of the fluorescence filter information set by the search setting section, and metadata corresponding to the single fluorescence images and metadata corresponding to the reference file; and a display control section configured to separate the single fluorescence images and the reference file searched by the search section and cause a display section to display a list of the separated single fluorescence images and reference file.

### CROSS-REFERENCE TO RELATED APPLICATIONS

The present application claims foreign priority based on Japanese Patent Application No. 2025-039961, filed Mar. 13, 2025, the contents of which are incorporated herein by reference.

### BACKGROUND OF THE INVENTION

### 1. Technical Field

The present disclosure relates to an information processing apparatus, an information processing method, and a program storage medium storing a program.

### 2. Description of the Related Art

For example, JP2007-316993A discloses an image processing apparatus capable of storing a plurality of pieces of image data and displaying images of the plurality of pieces of image data on a display section. In the image processing apparatus of JP2007-316993A, a file search display region is provided on a display screen for displaying an image. The file search display region shows a screen for inputting a file type, a file name, a keyword, and a file creation time as search conditions.

When a user performs a search execution operation after inputting a search condition, a search for a file matching the search condition is executed.

Meanwhile, there is a fluorescence microscope capable of performing fluorescence observation of an observation target by irradiating the observation target with excitation light and detecting fluorescence generated in the observation target. When observation is performed with such a fluorescence microscope, if a plurality of types of image files and files bundling image groups thereof can be searched and displayed together, convenience can be improved, but such a search function and a display function are not disclosed in JP2007-316993A.

### SUMMARY OF THE INVENTION

The present disclosure has been made in view of such a point, and an object of the present disclosure is to improve convenience at the time of observation by enabling cross-sectional searches of single fluorescence images and a reference file that bundles the single fluorescence images, and displaying a desired image.

In order to achieve the above object, according to one embodiment of the present disclosure, an information processing apparatus can be assumed. An information processing apparatus includes: a storage section configured to store a plurality of single fluorescence images acquired by different fluorescence filters, each of the single fluorescence images including metadata, and store a reference file that bundles some single fluorescence images from among the plurality of single fluorescence images; a search setting section configured to set fluorescence filter information as a search condition; a search section configured to search for the single fluorescence images and the reference file that satisfy the search condition on the basis of the fluorescence filter information set by the search setting section, and metadata corresponding to the single fluorescence images and metadata corresponding to the reference file stored in the storage section; and a display control section configured to separate the single fluorescence images and the reference file searched by the search section and cause a display section to display a list of the separated single fluorescence images and reference file.

According to this configuration, when the fluorescence filter information is set as the search condition, the single fluorescence images and the reference file bundling the single fluorescence images that satisfy the search condition are searched. In this manner, it is possible to cross-sectionally search the single fluorescence images and the reference file that bundles the single fluorescence images. Then, since the searched single fluorescence images and the reference file are displayed on the display section in a separated state, a desired image can be presented to a user. Note that, when the search section performs the search, the search may be performed on the basis of the metadata included in the reference file, or the search may be performed on the basis of the metadata included in the single fluorescence images corresponding to the reference file.

Furthermore, according to another embodiment of the present disclosure, an information processing method of searching for a fluorescence image of an observation target can be assumed. An information processing method includes the steps of: causing a storage section to store a plurality of single fluorescence images acquired by different fluorescence filters, each of the single fluorescence images including metadata, and causing the storage section to store a reference file that bundles some single fluorescence images from among the plurality of single fluorescence images; setting fluorescence filter information as a search condition in a search setting section; searching for the single fluorescence images and the reference file that satisfy the search condition in a search section on a basis of the fluorescence filter information set as the search condition, and metadata corresponding to the stored single fluorescence images and metadata corresponding to the stored reference file, and separating the searched single fluorescence images and reference file and causing a display section to display a list of the separated single fluorescence images and reference file.

Furthermore, according to still another embodiment of the present disclosure, a program causes a computer to execute the steps of: storing a plurality of single fluorescence images acquired by different fluorescence filters, each of the single fluorescence images including metadata, and storing a reference file that bundles some single fluorescence images from among the plurality of single fluorescence images; setting fluorescence filter information as a search condition; searching for the single fluorescence images and the reference file that satisfy the search condition on a basis of the fluorescence filter information set as the search condition, and metadata corresponding to the stored single fluorescence images and metadata corresponding to the stored reference file; and separating the searched single fluorescence images and reference file and causing a display section to display a list of the separated single fluorescence images and reference file, and a program storage medium storing the program is included.

As described above, it is possible to display a desired image by cross-sectionally searching the single fluorescence images and the reference file that bundles the single fluorescence images, and thus, convenience at the time of observation is improved.

### BRIEF DESCRIPTION OF THE DRAWINGS

FIG. 1 is a diagram illustrating a configuration of a fluorescence microscope according to an embodiment of the present invention;

FIG. 2 is a diagram describing an internal structure of a microscope body and a light source unit;

FIG. 3 is a schematic diagram illustrating an optical path of the fluorescence microscope;

FIG. 4 is a perspective view of a microscope body in which a base housing, a transmitted illumination section, a stage frame, an upper frame, and the like are omitted;

FIG. 5 is a plan view of a filter unit;

FIG. 6 is a perspective view of the filter unit as viewed from above;

FIG. 7 is a perspective view of a single filter cube as viewed from above;

FIG. 8 is a perspective view of the single filter cube as viewed from below;

FIG. 9 is a perspective view of an emission filter unit as viewed from above;

FIG. 10 is a cross-sectional view of a transmitted illumination body;

FIG. 11 is a diagram illustrating an example of a data structure of a single fluorescence image;

FIG. 12 is a diagram illustrating an example of a data structure of a group file;

FIG. 13 is a diagram illustrating an example of a data structure of an ICM file;

FIG. 14 is a diagram illustrating an example of a display screen;

FIG. 15 is a diagram illustrating an input example of a search condition;

FIG. 16 is a diagram illustrating an example of a user interface screen displaying a list of single fluorescence images;

FIG. 17 is a diagram illustrating an example of a user interface screen displaying a list of reference files;

FIG. 18 is a diagram illustrating an example in which metadata is displayed in a table format;

FIG. 19 is a diagram illustrating an example in which single fluorescence images are displayed in a thumbnail format;

FIG. 20 is a flowchart illustrating an example of a search process;

FIG. 21 is a flowchart illustrating details of a filtering process;

FIG. 22 is a flowchart illustrating details of a creation process of common format data;

FIG. 23 is a diagram illustrating an example of first common format data;

FIG. 24 is a diagram illustrating an example of second common format data;

FIG. 25 is a diagram illustrating an example of third common format data;

FIG. 26 is a diagram illustrating an example of a common format data set;

FIG. 27 is a diagram illustrating an example of a search result list;

FIG. 28 is a flowchart illustrating details of a display process; and

FIG. 29 is a diagram illustrating an example of an analysis application determination table.

### DETAILED DESCRIPTION

Hereinafter, embodiments of the present invention will be described in detail with reference to the drawings. Note that the following description of preferred embodiments is merely exemplary in nature and is not intended to limit the present invention, its application, or its use. For example, the relative size and positional relationship of each member illustrated in the drawings are for describing one embodiment, and do not limit the present invention.

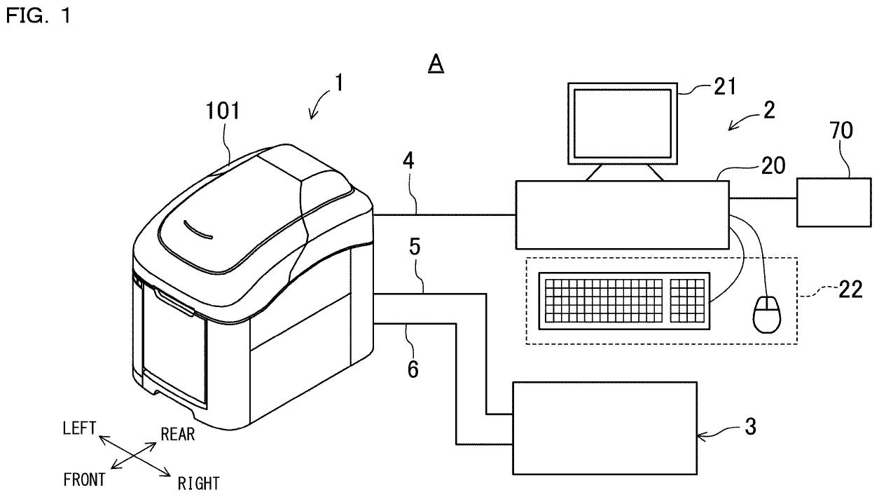

FIG. 1 is a diagram illustrating a configuration of a fluorescence microscope A according to an embodiment of the present invention. The fluorescence microscope A can perform fluorescence observation of an observation target. Furthermore, in addition to the fluorescence observation, the fluorescence microscope A can perform bright field observation, dark field observation, phase difference observation, differential interference observation, and the like using light transmitted through the observation target. In the following description, the bright field observation, the dark field observation, the phase difference observation, and the differential interference observation using light transmitted through the observation target are collectively referred to as transmission observation.

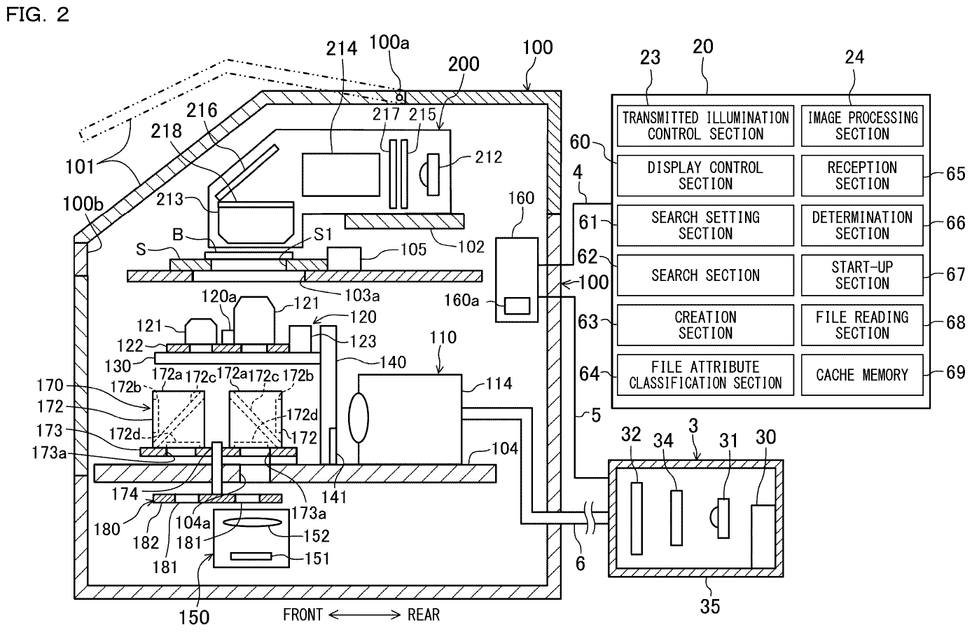

The observation target B (schematically illustrated in FIG. 2) observed in the present embodiment is a biological specimen or the like including various proteins. In a case where fluorescence observation of a biological specimen is performed, a fluorescent reagent that binds to a specific protein is used for the observation target B. Examples of the fluorescent reagent include green fluorescent protein (GFP), TexasRed, and diamidinophenylindole (DAPI), but fluorescence observation using a fluorescent reagent other than these is also possible.

For example, the GFP absorbs light having a wavelength of around 490 nm and emits light having a wavelength of around 510 nm. The TexasRed absorbs light having a wavelength of around 596 nm and emits light having a wavelength of around 620 nm. The DAPI absorbs light having a wavelength of around 345 nm and emits light having a wavelength of around 455 nm. As described above, the wavelength of light absorbed by the fluorescent reagent is different, and the wavelength of light emitted is also different.

The fluorescence microscope A includes, for example, a microscope body 1, an electronic computer 2, and a light source unit 3. Therefore, the fluorescence microscope A can also be referred to as a fluorescence microscope system or a fluorescence microscope device. Furthermore, the fluorescence microscope A has a search function for searching for a fluorescence image of the observation target B. The fluorescence microscope A having this search function can also be referred to as an information processing apparatus. By using the fluorescence microscope A, it is possible to execute an information processing method of searching for a fluorescence image of the observation target B.

The microscope body 1 and the electronic computer (computer) 2 are connected by a first wiring 4 and can communicate with each other. The microscope body 1 and the light source unit 3 are connected by a second wiring 5 and can communicate with each other. Moreover, the microscope body 1 and the light source unit 3 are connected by a fiber cable (transmission member) 6, and excitation light generated by the light source unit 3 can be transmitted to the microscope body 1 via the fiber cable 6 as described later. As described above, the fluorescence microscope A also includes the first wiring 4, the second wiring 5, and the fiber cable 6. As the first wiring 4, for example, a local area network (LAN) cable, a universal serial bus (USB) cable, or the like can be used. As the second wiring 5, for example, a cable in which a power supply line and a signal line are integrated can be used.

Note that the electronic computer 2 may be omitted, and the function of the electronic computer 2 may be incorporated in the microscope body 1. Furthermore, the light source unit 3 may be omitted, and the function of the light source unit 3 may be incorporated in the microscope body 1. Furthermore, the microscope body 1 may be configured by a single device.

As illustrated in FIG. 2, the light source unit 3 includes a power supply device 30, an illumination section 31, an excitation filter 32 that selectively transmits a wavelength of excitation light emitted from the illumination section 31, a light shielding mechanism 34, and an excitation illumination housing 35. The power supply device 30, the illumination section 31, the excitation filter 32, and the light shielding mechanism 34 are accommodated in the excitation illumination housing 35. A commercial power supply is connected to the power supply device 30, and power is supplied from the commercial power supply. The power supply device 30 supplies power supplied from the commercial power supply to the illumination section 31 and also supplies the power to the microscope body 1 via the second wiring 5. A power supply mode is not limited to the above-described mode, and the commercial power supply may be connected to the microscope body 1.

The illumination section 31 is a part that generates light used for observation, and includes, for example, a white light emitting diode (LED) or the like. The illumination section 31 may be configured with a light source other than a white light emitting diode (LED), and may be, for example, another light source such as a metal halide lamp, a mercury lamp, or a xenon lamp. The light emitted from the illumination section 31 is excitation light used at the time of fluorescence observation.

The light shielding mechanism 34 is, for example, a mechanical shutter. The light shielding mechanism 34 is disposed so as to be located on an optical path of light emitted from the illumination section 31. In a case where the light shielding mechanism 34 is in the non-light shielding state, the light emitted from the illumination section 31 passes through the light shielding mechanism 34 and enters the excitation filter 32. On the other hand, in a case where the light shielding mechanism 34 is in a light shielding state, the light emitted from the illumination section 31 is blocked and is not incident on the excitation filter 32. The light shielding mechanism 34 may include a light modulation element capable of switching between passage and blocking of light emitted from the illumination section 31. The light shielding mechanism 34 is in a non-light shielding state in a case where fluorescence observation of the observation target B is performed, and is in a light shielding state in a case where fluorescence observation of the observation target B is not performed.

The excitation filter 32 includes a plurality of excitation filters corresponding to the types of fluorescent reagents. When the light emitted from the illumination section 31 enters the excitation filter 32, the wavelength of the excitation light is selectively transmitted and emitted to one end portion of the fiber cable 6. The excitation light incident on one end portion of the fiber cable 6 is transmitted to the other end portion of the fiber cable 6 and is incident on the microscope body 1. The fiber cable 6 may be either a glass fiber or a quartz fiber. Furthermore, a liquid light guide or the like can also be used as the transmission member. Since the light source unit 3 is separated from the microscope body 1 in this manner, it is not necessary to provide the illumination section 31 that easily generates heat in the microscope body 1. Therefore, a countermeasure for heat dissipation of the microscope body 1 becomes unnecessary, and it is possible to achieve both irradiation of strong excitation light and miniaturization of the microscope body 1.

The microscope body 1 includes a base housing 100. Since the base housing 100 is separated from the excitation illumination housing 35, it can be installed separately from the excitation illumination housing 35. In the description of this embodiment, as illustrated in a front-rear direction and a left-right direction in FIG. 1, a side facing an observer (user) who performs observation using the microscope body 1 is referred to as a front side of the microscope body 1, a side opposite to the side facing the observer is referred to as a rear side of the microscope body 1, a right side as viewed from the observer is simply referred to as a right side, and a left side as viewed from the observer is simply referred to as a left side. The definitions of these directions are for convenience of description of the embodiment, and do not limit the present invention.

An opening 100b is formed in an upper portion of the base housing 100. An upper surface lid 101 for opening and closing the opening 100b is provided in the upper portion of the base housing 100. The rear side of the upper surface lid 101 is connected to the upper portion of the base housing 100 via a shaft 100a extending in the left-right direction. As indicated by a virtual line, the opening 100b of the base housing 100 can be opened by rotating the upper surface lid 101 upward around the shaft 100a. On the other hand, by closing the opening 100b of the base housing 100 as indicated by the solid line, the inside of the base housing 100 can be made a dark room.

The base housing 100 of the microscope body 1 includes an upper frame 102, a stage frame 103, and a lower frame 104. The upper frame 102 is provided in an upper portion inside the base housing 100. The stage frame 103 is provided below the upper frame 102 inside the base housing 100. The lower frame 104 is provided below the stage frame 103 inside the base housing 100. The upper frame 102, the stage frame 103, and the lower frame 104 extend horizontally and are in parallel positional relationship with each other. Openings 103a and 104a through which light used for observation of the observation target B passes are formed in the stage frame 103 and the lower frame 104, respectively. The opening 103a of the stage frame 103 and the opening 104a of the lower frame 104 are formed such that their centers are located on the same straight line extending in an up and down direction.

The microscope body 1 includes a stage S on which the observation target B is placed. The stage S is accommodated in the base housing 100 and is placed on the upper surface of the stage frame 103. The stage S also has an opening S1 through which light used for observation of the observation target B passes. The opening S1 of the stage S is disposed so as to overlap the opening 103a of the stage frame 103 in plan view. Moreover, an XY driving device 105 that drives the stage S in an X direction and a Y direction orthogonal to each other is attached to the upper surface of the stage frame 103. The X direction is the left-right direction, and the Y direction is the front-rear direction. That is, the XY driving device 105 is a device for moving the stage S in a horizontal direction.

The microscope body 1 includes an illumination module (illumination section) 110 that irradiates the observation target B placed on the stage S with excitation light as observation light. The illumination module 110 is accommodated in the base housing 100 and attached to the lower frame 104. In the present embodiment, the illumination module 110 is provided behind the optical module 120 described later. Note that the illumination module 110 may be provided below the optical module 120.

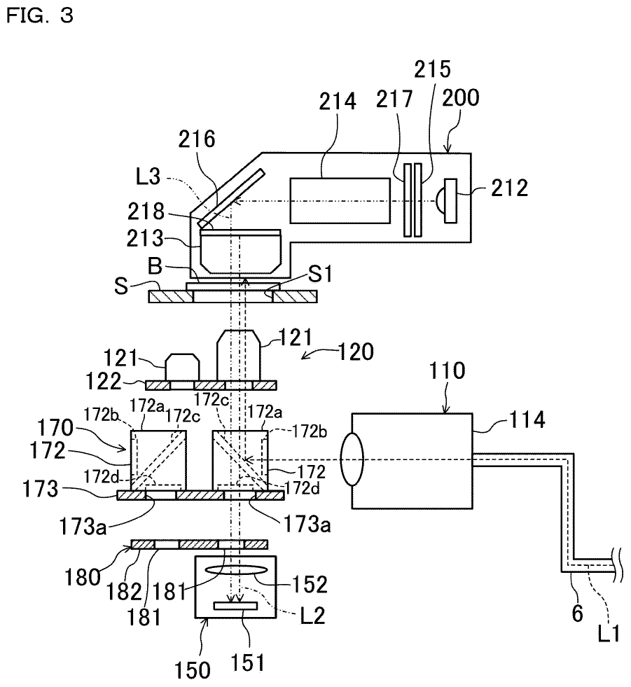

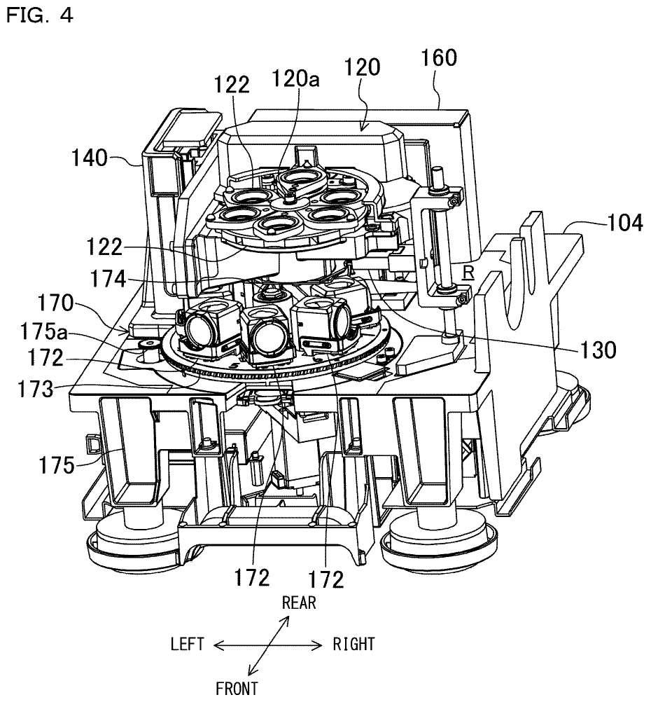

As illustrated in FIG. 3, the other end (the end on the side from which the excitation light is emitted) of the fiber cable 6 is connected to the rear side of the illumination module 110. In FIG. 3, a broken line L1 indicates an optical path of the excitation light.

The illumination module 110 is an excitation illumination section that irradiates the observation target B placed on the stage S with excitation light for fluorescence observation, and includes a module case 114. The other end portion of the fiber cable 6 is connected to the lower portion on the rear side of the module case 114. Furthermore, an optical modulation element that modulates the excitation light emitted from the fiber cable 6 may be provided in the module case 114.

The excitation light emitted from the module case 114 is emitted forward from the front side of the module case 114. The optical path of the excitation light emitted from the module case 114 is along the X direction.

The microscope body 1 includes an optical module 120, a Z-plate 130, and a Z-drive module 140. The optical module 120, the Z-plate 130, and the Z-drive module 140 are accommodated in the base housing 100. The optical module 120 is disposed on the optical path L1 of the excitation light emitted from the illumination module 110, collects the excitation light emitted from the illumination module 110, guides the excitation light to the observation target B, and collects the fluorescence generated in the observation target B. In FIG. 3, an optical path of fluorescence generated in the observation target B is indicated by a one-dot chain line L2. The optical module 120 is provided in front of the illumination module 110.

The Z-plate 130 is a member connected to the optical module 120, is provided between the stage frame 103 and the lower frame 104, and extends along the XY plane.

The optical module 120 includes a plurality of objective lenses 121, a revolver 122, and a revolver drive device 123. The number of the objective lenses 121 is not particularly limited, and may be any number. The objective lens 121 is disposed below the stage S, and is a member for collecting fluorescence generated from the observation target B by being irradiated with excitation light from the illumination module 110 at the time of fluorescence observation. Furthermore, at the time of transmission observation, the objective lens 121 collects the transmitted light emitted from the transmitted illumination section 200 to be described later and transmitted through the observation target B. The objective lens 121 is an observation optical system. The optical axis of each objective lens 121 is along the Z direction.

The revolver 122 is provided on the Z-plate 130 and is supported by the Z-plate 130 so as to be rotatable about a revolver shaft 120a extending in the vertical direction (Z direction). The plurality of objective lenses 121 is detachably attached to the revolver 122 in a state of being disposed so as to surround the revolver shaft 120a.

The revolver drive device 123 is attached to the upper surface of the Z-plate 130. By rotating the revolver 122 around the revolver shaft 120a by the revolver drive device 123, a desired one objective lens 121 among the plurality of objective lenses 121 is disposed immediately below the opening 103a of the stage frame 103, and the optical axis of the objective lens 121 is disposed on the optical path L2 of the fluorescence generated in the observation target B and the optical path L3 of the transmitted light. In this manner, the objective lens 121 used for observation can be switched by the revolver drive device 123. That is, the revolver 122 is a member in which the plurality of objective lenses 121 is disposed to be rotatable about the revolver shaft 120a and that switches the objective lens 121 disposed on the observation optical path.

The Z-drive module 140 is provided in a portion on the left side and the rear side of the lower frame 104. A lower portion of the Z-drive module 140 is attached to the upper surface of the lower frame 104, and the Z-drive module 140 has a shape protruding upward from the upper surface of the lower frame 104.

The Z-drive module 140 is connected to the Z-plate 130 and moves the objective lens 121, the revolver 122, and the revolver drive device 123 of the optical module 120 connected to the Z-plate 130 in the Z-direction. The Z-drive module 140 includes an electric adjustment mechanism 141. By driving the Z-plate 130 in the Z direction by the electric adjustment mechanism 141, the relative distance in the Z direction between the objective lens 121 used for observation and the observation target B is adjusted.

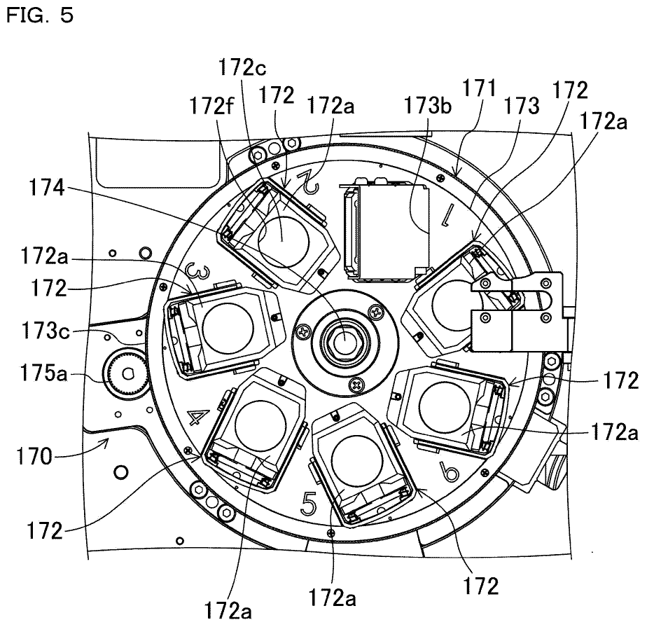

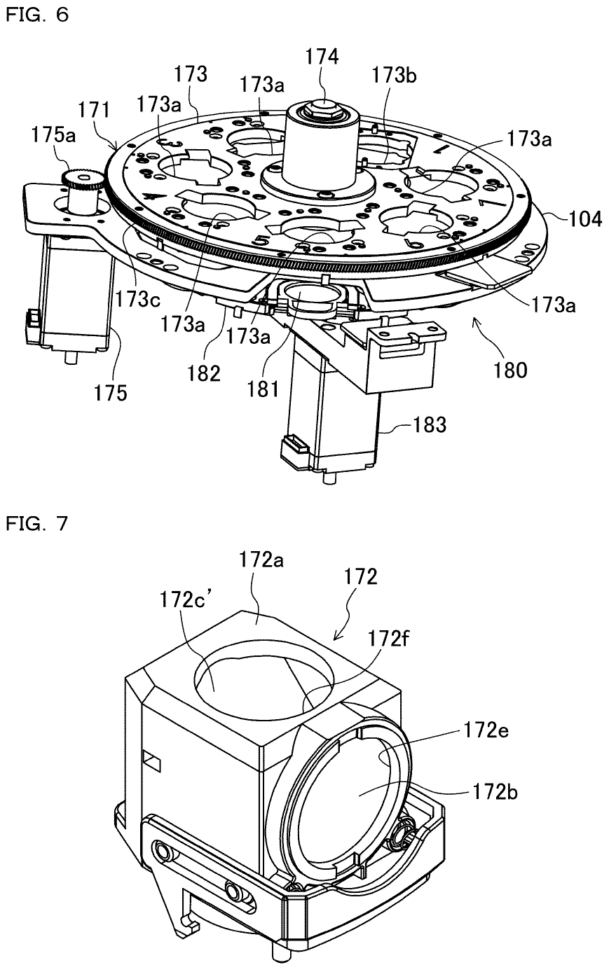

As illustrated in FIGS. 5 and 6, the microscope body 1 includes a filter unit 170 accommodated in the base housing 100. The filter unit 170 is disposed below the revolver 122 and includes a filter switching device 171 and a plurality of filter cubes 172. The filter switching device 171 includes a filter turret 173 to which a plurality of filter cubes 172 is detachably attached, a support shaft 174 that supports the filter turret 173, and a turret drive motor 175.

The support shaft 174 extends in the up and down direction. A portion intermediate in the up and down direction of the support shaft 174 is attached to the lower frame 104. The upper portion of the support shaft 174 protrudes upward from the upper surface of the lower frame 104. A central portion of the filter turret 173 is supported on an upper portion of the support shaft 174 so as to be relatively rotatable.

As illustrated in FIG. 6, the filter turret 173 has a disk shape. In the filter turret 173, a plurality of through holes 173a and 173b penetrating in the up and down direction is formed at intervals in a circumferential direction of the support shaft 174. The intervals between the through holes 173a and 173b in the circumferential direction are equal. In this embodiment, the seven through holes 173a and 173b are formed, and the filter cube 172 can be attached to the six through holes 173a among them, while the filter cube 172 cannot be attached to one through hole 173b.

The through hole 173b formed in the filter turret 173 is a hole for passing light transmitted through the observation target B at the time of transmission observation. At the time of transmission observation, the filter turret 173 is rotated such that the through hole 173b is disposed on the optical path L3 of the light transmitted through the observation target B. In this manner, by rotating the filter turret 173, it is possible to switch between a state (inserted state) in which the filter cube 172 and the absorption filter 172d included in the filter cube 172 are disposed on the observation optical path and a state (removed state) in which the filter cube 172 and the absorption filter 172d included in the filter cube 172 are not disposed. That is, the filter turret 173 is a member that inserts and removes the filter cube 172 and the absorption filter 172d included in the filter cube 172 on the observation optical path.

A large number of driven teeth 173c are provided in the circumferential direction on the outer peripheral portion of the filter turret 173. The turret drive motor 175 is provided with a pinion gear 175a that meshes with the driven teeth 173c. When the turret drive motor 175 is activated to rotate the pinion gear 175a, the filter turret 173 can be rotated about the support shaft 174. The rotation angle of the filter turret 173 can be any rotation angle by controlling the turret drive motor 175.

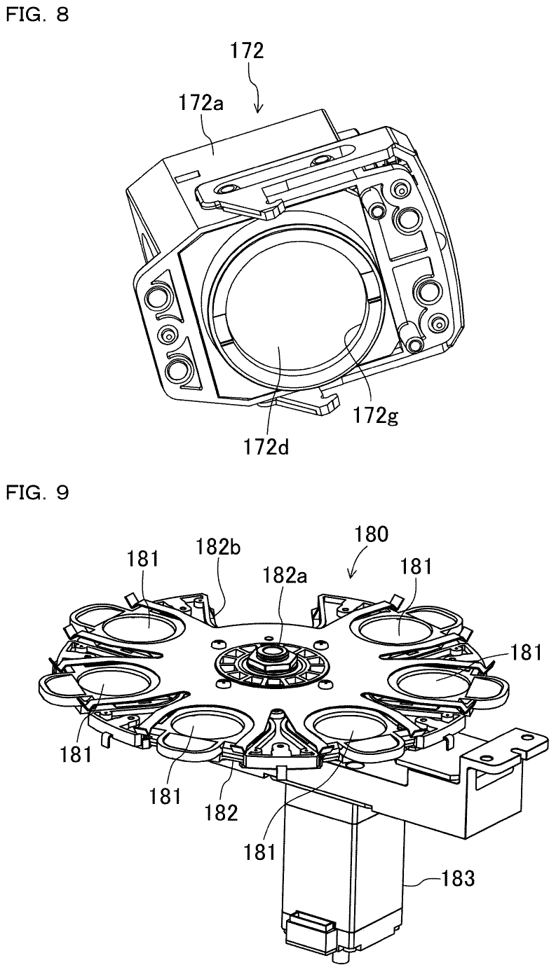

As illustrated in FIGS. 7 and 8, each filter cube 172 includes a frame 172a having an aerial structure, an excitation filter 172b, a dichroic mirror 172c, and an absorption filter 172d. The excitation filter 172b and the absorption filter 172d are examples of a fluorescence filter.

The frame 172a is a cubic member that supports the excitation filter 172b, the dichroic mirror 172c, and the absorption filter 172d. Since each filter cube 172 is disposed below the revolver 122, the excitation filter 172b, the dichroic mirror 172c, and the absorption filter 172d are also disposed below the revolver 122. Since a plurality of the filter cubes 172 is provided, a plurality of excitation filters 172b, a plurality of the dichroic mirrors 172c, and a plurality of the absorption filters 172d are also provided corresponding to the number of filter cubes 172.

The frame 172a includes an entrance window 172e through which the excitation light from the illumination module 110 is incident, an upper emission window 172f through which the excitation light is emitted, and a lower emission window 172g through which the fluorescence generated in the observation target B is emitted. The entrance window 172e is formed on a side surface of the frame 172a, and an excitation filter 172b is provided in the entrance window 172e. The upper emission window 172f is formed on the upper surface of the frame 172a. The lower emission window 172g is formed on the lower surface of the frame 172a, that is, a surface opposed to the surface in the up and down direction on which the upper emission window 172f is formed. The absorption filter 172d is provided in the lower emission window 172g. The absorption filter 172d is an optical member that selectively transmits fluorescence in a predetermined wavelength band among fluorescence collected by the objective lens 121 at the time of fluorescence observation. That is, in the plurality of absorption filters 172d, wavelengths to be transmitted among the fluorescence generated in the observation target B irradiated with the excitation light by the illumination section 31 are different from each other.

A multi-dichroic mirror 172c′ that transmits fluorescence in a plurality of wavelength bands may be provided as the dichroic mirror 172c inside the frame 172a. The dichroic mirror 172c is fixed to the frame 172a in a state of being inclined by a predetermined angle with respect to the Z direction. The excitation light transmitted through the excitation filter 172b provided in the entrance window 172e is reflected upward by the dichroic mirror 172c, passes through the upper emission window 172f, and is emitted upward from the frame 172a. The excitation light having passed through the upper emission window 172f passes through the objective lens 121 and is applied to the observation target B on the stage S.

Since the plurality of filter cubes 172 is attached to the filter turret 173, the plurality of excitation filters 172b, the plurality of dichroic mirrors 172c, and the plurality of absorption filters 172d are disposed in the filter turret 173. By rotating the filter turret 173, the filter cube 172 used at the time of fluorescence observation can be switched to arbitrary one filter cube 172 among the plurality of filter cubes 172. The filter turret 173 is a member in which the plurality of excitation filters 172b, the plurality of dichroic mirrors 172c, and the plurality of absorption filters 172d are rotatably disposed about the support shaft 174 extending in the vertical direction and that switches the excitation filters 172b, the dichroic mirrors 172c, and the absorption filters 172d disposed on the observation optical path.

The microscope body 1 includes a control board 160. The control board 160 is a part that controls each part of the microscope body 1, and includes, for example, a turret control section 160a that controls the filter turret 173. The turret control section 160a is connected to the turret drive motor 175 and controls the turret drive motor 175. By controlling the turret drive motor 175 by the turret control section 160a, the filter turret 173 can be rotated to a desired rotation angle and stopped, whereby one absorption filter 172d disposed on the observation optical path can be sequentially switched. That is, the turret control section 160a is a part that sequentially switches one absorption filter 172d disposed on the observation optical path by the filter turret 173.

One set is formed by the excitation filter 172b, the dichroic mirror 172c, and the absorption filter 172d provided in one filter cube 172. The excitation filter 172b, the dichroic mirror 172c, and the absorption filter 172d provided in each filter cube 172 are different so as to correspond to the fluorescent reagent used at the time of fluorescence observation. In a case where, for example, GFP, TexasRed, DAPI, or the like is used as the fluorescent reagent, the filter cube 172 corresponding to GFP, TexasRed, DAPI, or the like is attached to the filter turret 173.

The excitation filter 172b of the filter cube 172 corresponding to the GFP allows light having a wavelength of around 490 nm to pass therethrough, and the absorption filter 172d allows light having a wavelength of around 510 nm to pass therethrough. The dichroic mirror 172c reflects light having a wavelength of around 490 nm and allows light having a wavelength of around 510 nm to pass therethrough.

Furthermore, the excitation filter 172b of the filter cube 172 corresponding to the TexasRed allows light having a wavelength of around 596 nm to pass therethrough, and the absorption filter 172d allows light having a wavelength of around 620 nm to pass therethrough. The dichroic mirror 172c reflects light having a wavelength of around 596 nm and allows light having a wavelength of around 620 nm to pass therethrough.

Furthermore, the excitation filter 172b of the filter cube 172 corresponding to the DAPI allows light having a wavelength of around 345 nm to pass therethrough, and the absorption filter 172d allows light having a wavelength of around 455 nm to pass therethrough. The dichroic mirror 172c reflects light having a wavelength of around 345 nm and allows light having a wavelength of around 455 nm to pass therethrough.

An emission filter unit 180 as illustrated in FIG. 9 is provided below the filter turret 173, that is, below the dichroic mirror 172c. The emission filter unit 180 includes a plurality of emission filters 181 having mutually different wavelengths to be transmitted among the fluorescence transmitted through the dichroic mirror 172c, a filter wheel 182 to which the plurality of emission filters 181 is detachably attached, and a wheel drive motor 183. Although not illustrated, the wheel drive motor 183 transmits a rotational force to the filter wheel 182 via, for example, a gear. The emission filter 181 is an example of a fluorescence filter.

The plurality of emission filters 181 is disposed at intervals in the circumferential direction of the filter wheel 182. By rotating the filter wheel 182, arbitrary one emission filter 181 of the plurality of emission filters 181 can be disposed on the optical path L2 of fluorescence. The filter wheel 182 is provided with a notch 182b to which the emission filter 181 is not attached. The notch 182b is a part through which the light transmitted through the observation target B passes at the time of transmission observation, and the filter wheel 182 is rotated so that the notch 182b is disposed on the optical path L3 of the light transmitted through the observation target B at the time of transmission observation.

The emission filter 181 is a member for selectively transmitting fluorescence in a specific wavelength band. That is, the plurality of emission filters 181 functions as a multi-filter that transmits fluorescence in a plurality of wavelength bands by sequentially switching the absorption filters disposed on the optical path L2 of fluorescence. For example, the wavelength band of the received fluorescence can be changed by fixing the filter turret 173 in which the multi-band dichroic mirror 172c that transmits fluorescence of a plurality of wavelength bands is disposed and rotating the filter wheel 182 to sequentially switch the emission filter 181.

As illustrated in FIGS. 2 and 3, the microscope body 1 includes a transmitted illumination section 200 that irradiates the observation target B placed on the stage S with observation light. The transmitted illumination section 200 includes a transmitted illumination body 210. The transmitted illumination body 210 includes a transmitted illumination housing 211, and the transmitted illumination housing 211 has a cylindrical shape elongated in the front-rear direction.

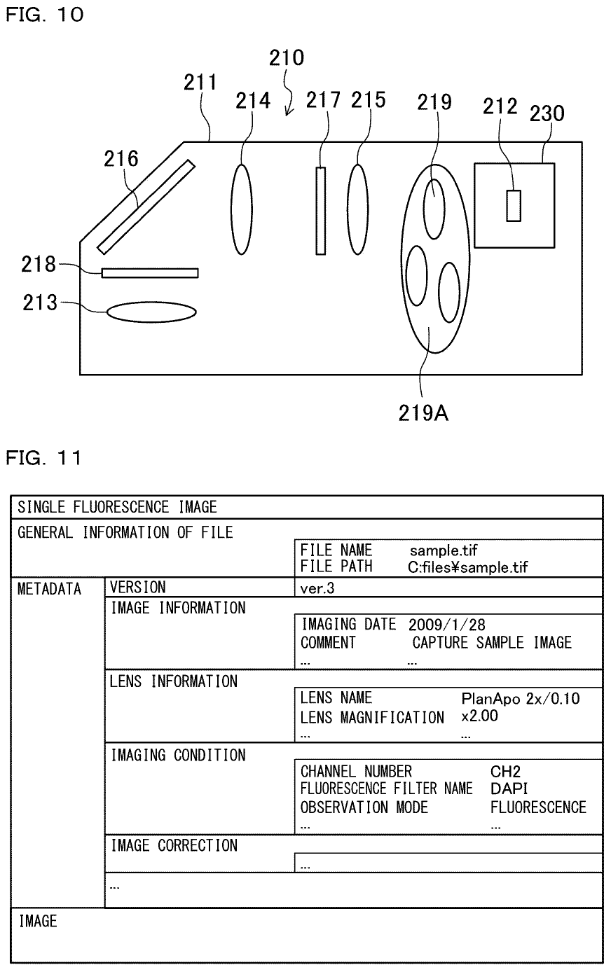

As illustrated in FIG. 10, the transmitted illumination body 210 includes a white light source 212 that emits transmitted illumination light that is illumination light for transmission observation, a filter 219, a condenser lens 213, a relay lens 214, and an illumination lens 215. Here, the filter 219 may be a plurality of filters having transmission bands of a red band, a green band, and a blue band. In this case, each filter is attached to the wheel 219A, and the wavelength with which the observation target B is irradiated can be switched by rotating the wheel 219A. Furthermore, the filter 219 may be an optical element capable of electrically switching the transmission band.

As illustrated in FIG. 10, the illumination lens 215 is disposed in front of a light source accommodation portion 230. Therefore, red light, green light, and blue light sequentially generated by being emitted from the white light source 212 and being transmitted through the filter 219 are incident on the illumination lens 215. The illumination lens 215 is a member for collecting light of different wavelengths sequentially generated by being emitted from the white light source 212 and being transmitted through the filter 219.

The relay lens 214 is a member disposed between the condenser lens 213 and the white light source 212 and having a focal plane at an aperture diaphragm of the condenser lens 213 and a rear focal position of the illumination lens 215. The optical axis of the relay lens 214 and the optical axis of the illumination lens 215 are both oriented in the front-rear direction and coincide with each other.

The transmitted illumination body 210 includes a mirror 216. The mirror 216 is provided in a front portion of the transmitted illumination housing 211. The light emitted forward from the relay lens 214 is incident on the mirror 216 and is reflected downward by the mirror 216.

The condenser lens 213 is a member that is disposed below the mirror 216, collects the light emitted from the white light source 212, and irradiates the observation target B placed on the stage S with transmitted illumination light. The optical axis of the condenser lens 213 is directed in the up and down direction, and the light reflected downward by the mirror 216 is incident on the condenser lens 213 and then exits downward from the condenser lens 213. In such an optical system, the illumination lens 215 is designed to focus the light from the white light source 212 at a position conjugate with the aperture diaphragm position of the relay lens 214.

A first light diffusion plate 217 as a first light homogenizing element is provided at a rear focal position of the illumination lens 215. Moreover, a second light diffusion plate 218 as a second light homogenizing element is provided at the position of the aperture diaphragm of the condenser lens 213. Since the first light diffusion plate 217 and the second light diffusion plate 218 are provided on the optical path of the transmitted illumination light, light beams can be scattered and mixed. Therefore, unevenness of the light emitted from the condenser lens 213 is eliminated, and the light can be brought close to uniform light.

The optical path L3 (illustrated in FIG. 3) of the transmitted light partially overlaps with the optical path L1 of the excitation light. That is, the excitation light is emitted from below to above the stage S. A part of the excitation light transmitted through the observation target B is incident on the condenser lens 213 of the transmitted illumination section 200.

The microscope body 1 includes an imaging module 150. The imaging module 150 is accommodated in the base housing 100 and is provided below the lower frame 104. The imaging module 150 is disposed on the optical path L2 of the fluorescence collected by the optical module 120 and detects the fluorescence collected by the optical module 120, and is disposed on the optical path L3 of the light transmitted through the observation target B and detects the light transmitted through the observation target B. In the present embodiment, the imaging module 150 is provided below the optical module 120.

The imaging module 150 includes a camera 151 and an imaging lens 152. The camera 151 is, for example, a complementary metal oxide semiconductor (CMOS) image sensor including an imaging element. The imaging element is, for example, a monochrome CMOS, and the camera 151 includes a monochrome camera including a monochrome CMOS. The imaging element may be another imaging element such as a charge coupled device (CCD) image sensor.

Fluorescence or transmitted light incident on the imaging module 150 is collected and imaged by the imaging lens 152, and then detected (received) by the camera 151. Therefore, an image of the observation target B is obtained. An analog electric signal (Hereinafter, referred to as a light reception signal.) corresponding to the amount of received light is output from each pixel of the imaging element of the camera 151 to a control board 160. The camera 151 is an example of a detection section that detects light collected by the objective lens 121 constituting the observation optical system.

Unlike the color CCD, the monochrome CCD constituting the camera 151 of the present embodiment does not need to be provided with a pixel that receives light of a red wavelength, a pixel that receives light of a green wavelength, and a pixel that receives light of a blue wavelength. Therefore, the measurement resolution of the monochrome CCD is higher than the resolution of the color CCD. Furthermore, unlike the color CCD, the monochrome CCD does not need to include a color filter in each pixel. Therefore, the sensitivity of the monochrome CCD is higher than the sensitivity of the color CCD. For these reasons, the camera 151 according to the present embodiment includes a monochrome CCD.

An analog/digital converter (A/D converter), a first in first out (FIFO) memory, and the like (not illustrated) are mounted on the control board 160. The light reception signal output from the camera 151 is sampled at a constant sampling period by the A/D converter and converted into a digital signal on the basis of the control of the electronic computer 2 illustrated in FIG. 1. The digital signals output from the A/D converter are sequentially accumulated in the FIFO memory. The digital signals accumulated in the FIFO memory are sequentially transferred to the electronic computer 2 as pixel data.

As illustrated in FIG. 1, the electronic computer 2 includes, for example, a desktop personal computer or a notebook personal computer, and includes a computer body 20, a display section 21, and an operation section 22. The computer body 20 includes a central processing unit (CPU), a read only memory (ROM), a random access memory (RAM), a storage device, and the like. The ROM stores a system program. The RAM stores various data and functions as a work region of the CPU. The storage device includes a solid state drive, a hard disk drive, or the like. The storage device stores a control program and an image processing program of the fluorescence microscope A. On the basis of the control program and the image processing program, the computer body 20 controls each part of the microscope body 1 and controls the light source unit 3. That is, the control program and the image processing program stored in the storage device are programs that cause a computer to execute a plurality of steps described later. The present invention also includes, for example, an optical disk, a magnetic disk, and the like as a program storage medium storing the programs. Furthermore, the storage device is also used to store various data such as image data output from the imaging module 150.

The display section 21 includes, for example, an LCD panel, an organic electroluminescence (EL) panel, or the like. On the display section 21, a user interface screen for performing various settings of the fluorescence microscope A is displayed, and an observation image or the like based on the image data output from the imaging module 150 is displayed.

The operation section 22 includes a member for operating the computer body 20. Examples of the member constituting the operation section 22 include a keyboard and a pointing device. Examples of the pointing device include a mouse and a joystick.

The computer body 20 controls each part of the microscope body 1, so that fluorescence observation using fluorescence can be performed and transmission observation can also be performed. At the time of fluorescence observation, the filter switching device 171 operates and switches to the filter cube 172 corresponding to the fluorescent reagent to be used. Furthermore, the objective lens is switched to a desired objective lens 121 by the revolver drive device 123. Moreover, in a case where the emission filter 181 is used, the emission filter unit 180 operates and is switched to a desired emission filter 181.

The light source unit 3 generates excitation light. The excitation light generated by the light source unit 3 is transmitted to the microscope body 1 via the fiber cable 6, transmitted through the filter cube 172 and the objective lens 121, and then applied to the observation target B. At the time of observation, the Z-drive module 140 operates, and focus adjustment is automatically executed.

The fluorescence generated in the observation target B is incident on the objective lens 121, then is transmitted through the filter cube 172 and the emission filter 181, and is incident on the imaging module 150. When the filter turret 173 is controlled by the turret control section 160a, the filter turret 173 switches the filter cube 172 disposed on the observation optical path. Since the filter cube 172 is sequentially switched by this switching operation, the fluorescence transmitted through one absorption filter 172d disposed on the observation optical path can be sequentially received by the camera 151. In this way, a plurality of single fluorescence images acquired by different fluorescence filters can be acquired.

Furthermore, it is also possible to sequentially switch the emission filter 181 in a state where arbitrary one dichroic mirror 172c is disposed on the observation optical path by the filter turret 173 controlled by the turret control section 160a. The wavelength band of the fluorescence detected by the camera 151 can also be sequentially switched by sequentially switching the emission filter 181. Also in this case, a plurality of single fluorescence images acquired by different fluorescence filters can be acquired.

The fluorescence incident on the imaging module 150 is received by the camera 151. The analog electric signal corresponding to the amount of received light is output from the control board 160 to the computer body 20 to generate image data, and a fluorescence image is displayed on the display section 21.

At the time of transmission observation, the observation target B is irradiated with the transmitted light emitted from the transmitted illumination section 200. In the transmission observation, the filter switching device 171 operates, and the filter cube 172 is not disposed on the optical path L3 of the transmitted light. Furthermore, the objective lens is switched to a desired objective lens 121 by the revolver drive device 123. Moreover, the emission filter unit 180 operates, and the emission filter 181 is not disposed on the optical path L3 of the transmitted light. Also at the time of transmission observation, the Z-drive module 140 operates, and focus adjustment is automatically executed. At the time of transmission observation, the camera 151 can receive the transmitted light transmitted through the observation target B in a state where the absorption filter 172d is disposed outside the observation optical path by the filter turret 173.

Specifically, the transmitted light transmitted through the observation target B is incident on the objective lens 121, and then is incident on the imaging module 150 without being transmitted through the filter cube 172 and the emission filter 181. The transmitted light incident on the imaging module 150 is received by the camera 151. An analog electric signal corresponding to the amount of received light is output from the control board 160 to the computer body 20. The computer body 20 is provided with an image processing section 24. The image processing section 24 generates a non-fluorescence image including, for example, a phase difference image, a bright field image, and the like on the basis of the transmitted light detected by the camera 151. In this manner, the image processing section 24 generates both the phase difference image and the bright field image as the non-fluorescence image.

The non-fluorescence image generated by the image processing section 24 is displayed on the display section 21. Note that the phase difference image is an image that can visualize the transparent structure of the unstained observation target B by utilizing a difference in refractive index of the observation target B. Furthermore, the bright field image is an image in which the structure can be viewed with the color or shade of the observation target B itself by directly observing the transmitted light passing through the objective lens 121.

In the present embodiment, both the fluorescence and the transmitted light are received by the common camera 151, but the present invention is not limited thereto, and as a detection section of light, a first detection section that sequentially receives the fluorescence transmitted through one absorption filter 172d disposed on the observation optical path by the turret control section 160a, and a second detection section that receives the transmitted light of the observation target B irradiated with the illumination light may be included.

The fluorescence microscope A of the present embodiment has a color observation function of generating a color image of the observation target B without using a color image generation filter applied at the time of conventional color image generation. The color observation function of the fluorescence microscope A can be realized by the white light source 212, the filter 219, and the transmitted illumination control section 23 and the image processing section 24 illustrated in FIG. 2.

The transmitted illumination control section 23 controls the white light source 212 of the transmitted illumination section 200 to emit light. Furthermore, the transmitted illumination control section 23 sequentially switches, in a time division manner, the wavelength of the light with which the observation target B is irradiated by driving the wheel 219A holding the filter 219. Specifically, the transmitted illumination control section 23 drives the wheel 219A holding the filter 219 so as to transmit only the light of the first wavelength, thereby irradiating the observation target B with the red light. Furthermore, the transmitted illumination control section 23 drives the wheel 219A holding the filter 219 so as to transmit only the light of the second wavelength, thereby irradiating the observation target B with the green light. Moreover, the transmitted illumination control section 23 drives the wheel 219A holding the filter 219 so as to transmit only the light of the third wavelength, thereby irradiating the observation target B with blue light. As described above, the transmitted illumination control section 23 drives the wheel 219A holding the filter 219 to sequentially switch, in a time division manner, the wavelength of the light with which the observation target B is irradiated, and controls the transmitted illumination section 200 so as to emit light having wavelengths different from each other. Here, although not illustrated, in a case where an optical element capable of electrically switching the transmission band is used as the filter, the transmitted illumination control section 23 irradiates the observation target B with red light by driving the filter to transmit only light of the first wavelength. Furthermore, the transmitted illumination control section 23 drives the filter so as to transmit only the light of the second wavelength, thereby irradiating the observation target B with the green light. Moreover, the transmitted illumination control section 23 drives the filter so as to transmit only the light of the third wavelength, thereby irradiating the observation target B with blue light. As described above, the transmitted illumination control section 23 drives the wheel 219A on which the filter 219 is held or the filter using the optical element capable of electrically switching the transmission band, thereby sequentially switching, in a time division manner, the wavelength of the light with which the observation target B is irradiated, and controlling the transmitted illumination section 200 so as to emit light having wavelengths different from each other. Note that the order of red light irradiation, blue light irradiation, and green light irradiation is not particularly limited.

In the case of red light irradiation, the red light transmitted through the observation target B is received by the camera 151. Thereafter, when blue light irradiation is performed, the blue light transmitted through the observation target B is received by the camera 151. Thereafter, when green light irradiation is performed, the green light transmitted through the observation target B is received by the camera 151. In this manner, the camera 151 sequentially receives transmitted light of different wavelengths transmitted through the observation target B.

The image processing section 24 is a part that generates a color image that is an observation image on the basis of transmitted light of different wavelengths sequentially received by the camera 151. That is, an analog electrical signal corresponding to the amount of received red light when the red light transmitted through the observation target B is received by the camera 151, an analog electrical signal corresponding to the amount of received blue light when the blue light transmitted through the observation target B is received by the camera 151, and an analog electrical signal corresponding to the amount of received green light when the green light transmitted through the observation target B is received by the camera 151 are transmitted to the image processing section 24. The image processing section 24 synthesizes three monochrome images generated by the received three analog electrical signals to generate a color image.

Next, a case of generating a monochrome image will be described. In a case of generating a monochrome image, the transmitted illumination control section 23 turns on the white light source 212 and irradiates the observation target B with white light. Furthermore, the transmitted illumination control section 23 transmits the white light emitted from the white light source 212 by rotating the wheel 219A having an empty hole in which the filter 219 is not disposed and driving the wheel 219A such that the empty hole is disposed on the optical path. Furthermore, in the case of a filter that electrically switches the transmission band, a mechanism that inserts and removes the filter onto and from the optical path may be further provided, and the observation target B may be irradiated with white light emitted from the white light source 212 by driving the filter to be removed from the optical path. The image processing section 24 generates a monochrome image on the basis of transmitted light of white light received by the camera 151.

The image processing section 24 can also generate a pseudo color image. In a case where the image processing section 24 generates the pseudo color image, the fluorescence sequentially detected by the camera 151 and the wavelength band of the absorption filter 172d disposed on the observation optical path are acquired. The image processing section 24 generates a plurality of pseudo color images on the basis of the fluorescence sequentially detected by the camera 151 and the wavelength band of the absorption filter 172d disposed on the observation optical path.

Furthermore, a plurality of single fluorescence images can be acquired by a Z stack that acquires a plurality of images with different imaging heights. Furthermore, a plurality of single fluorescence images can be acquired also when image stitching is performed in which a plurality of images is acquired and stitched by moving the observation target B in the XY direction. Moreover, even when time lapse imaging for acquiring a plurality of images at time intervals is performed, a plurality of single fluorescence images can be acquired.

Furthermore, for example, as in a case where the observation target B is accommodated in each cell of a multi-well, it is also possible to simultaneously capture a large number of observation targets B using a so-called image cytometer. In the image cytometer, similar imaging is repeated at a plurality of places, and the acquired data is analyzed collectively.

### (Image Search Function)

As illustrated in FIG. 2, the fluorescence microscope A includes a display control section 60, a search setting section 61, a search section 62, a creation section 63, a file attribute classification section 64, a reception section 65, a determination section 66, a start-up section 67, and a file reading section 68 as portions that execute generation of various user interface screens for realizing an image search function, display control of various user interface screens, control of each section, and various processes. The display control section 60, the search setting section 61, the search section 62, the creation section 63, the file attribute classification section 64, the reception section 65, the determination section 66, the start-up section 67, and the file reading section 68 can be configured by a computer body 20 of the electronic computer 2. For example, the display control section 60, the search setting section 61, the search section 62, the creation section 63, the file attribute classification section 64, the reception section 65, the determination section 66, the start-up section 67, and the file reading section 68 can be configured by a combination of hardware of the computer body 20 and a program executed by the computer body 20.

An image that can be a search target in the present search function can be stored in a storage section 70 (illustrated in FIG. 1) of the electronic computer 2. The storage section 70 includes, for example, a solid state drive, a hard disk drive, or the like. The storage section 70 may be installed in a place different from the place where the microscope body 1 is installed, and may be formed of, for example, an external medium or the like.

The storage section 70 stores a plurality of single fluorescence images acquired by different fluorescence filters, and stores a reference file that bundles some single fluorescence images among the plurality of single fluorescence images. The information processing method according to the present embodiment includes a step of storing a plurality of single fluorescence images and reference files in the storage section 70.

Although details will be described later, metadata including a single imaging condition and the like is associated with each single fluorescence image. Therefore, the storage section 70 stores a plurality of single fluorescence images each having metadata.

Examples of the file type of the single fluorescence image include “.tif”, “.jpg”, and the like in the case of one image file, “.ome.tif”, and the like in the case of an image file that can have a plurality of dimensions, and “.tile”, and the like in the case of a tiling image file. An image file that can have a plurality of dimensions has a plurality of images captured in different dimensions as one file. The tiling image file is obtained by performing image processing on a stitched image to form one stitched image.

The reference file bundles the output image files and has cross-sectional information thereof. An example of the reference file is, for example, a group file (group), and the group file is output by multidimensional imaging. The group file is a file in which one image file “.tif”, “.jpg”, image files “.ome.tif” that can have a plurality of dimensions, and the like are bundled.

Another example of a reference file is, for example, an ICM file (.icm), which is output at an image cytometer. The ICM file is a file that bundles group files (.group). That is, the reference file includes an ICM file as a first reference file that bundles a plurality of single fluorescence images acquired at different positions, and a group file as a second reference file that bundles a plurality of single fluorescence images. The ICM file as a first reference file is a file that bundles group files as second reference files that bundle a plurality of single fluorescence images acquired at each position.

FIG. 11 illustrates a data structure of a single fluorescence image. For example, general information of a file and metadata are associated with the single fluorescence image. The general information of the file of the single fluorescence image includes, for example, a file name, a file path, and the like. The metadata of the single fluorescence image includes, for example, version information, image information, lens information (information of the objective lens 121), imaging conditions, image correction information, and the like. The image information includes an imaging date, a comment input by the user, and the like. The comment can be freely input by the user through the user interface screen. The lens information includes a lens name used for imaging, a lens magnification, and the like. The imaging conditions include the number of the observation channel, the fluorescence filter name (fluorescence filter information), the observation mode, and the like. The observation channel includes a channel for acquiring bright field image data, a channel for acquiring fluorescence image data, a channel for acquiring phase difference image data, and the like, and can be specified by a number. The observation mode similarly includes modes such as bright field observation, fluorescence observation, and phase difference observation.

In a case where the filter wheel 182 is not used, a plurality of single fluorescence images can be acquired by switching a plurality of filter cubes 172. On the other hand, in a case where the filter wheel 182 is used in a channel for acquiring fluorescence image data, a plurality of single fluorescence images can be acquired by rotating the filter wheel 182 to switch the emission filter 181. The fluorescence filter information used when the single fluorescence image is acquired is included in the metadata of the single fluorescence image.

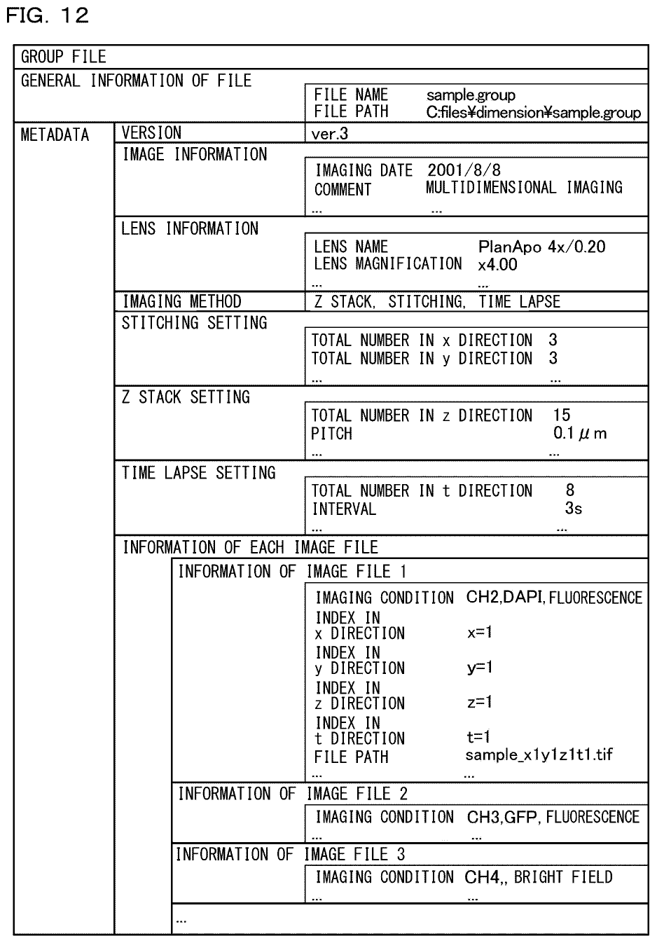

On the other hand, FIG. 12 illustrates a data structure of the group file included in the reference file. In the group file, for example, general information of the file and metadata are associated with each other. The general information of the group file includes, for example, a file name, a file path, and the like. The metadata of the group file includes, for example, version information, image information, lens information used for imaging, an imaging method, image stitching setting, Z stack setting, time lapse setting, information of each image file, and the like.

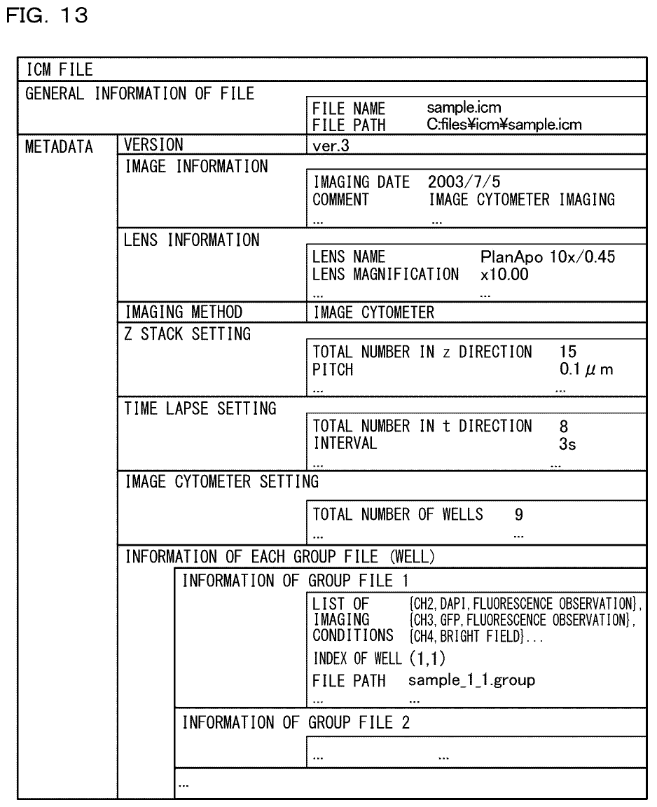

FIG. 13 illustrates a data structure of the ICM file included in the reference file. In the ICM file, for example, general information of the file and metadata are associated with each other. The general information of the ICM file includes, for example, a file name, a file path, and the like. The metadata of the ICM file includes, for example, version information, image information, lens information used for imaging, an imaging method, Z stack setting, time lapse setting, image cytometer setting, information of each group file (well), and the like.

The metadata corresponding to the reference file includes, for example, lens information used for imaging, an imaging method, a Z stack setting, a time lapse setting, and the like as a plurality of imaging conditions based on imaging conditions included in each single fluorescence image bundled by the reference file.

As illustrated in FIGS. 11 to 13, when the three data structures of the single fluorescence image, the group file, and the ICM file are compared, the metadata regions have different formats, but not all of the metadata regions are different, but some of the metadata regions are different. Specifically, in the metadata, version information, image information, and lens information used for imaging are common to three data structures of a single fluorescence image, a group file, and an ICM file. Furthermore, when the two data structures of the group file and the ICM file are compared, the imaging method, the Z stack setting, and the time lapse setting of the metadata are common.

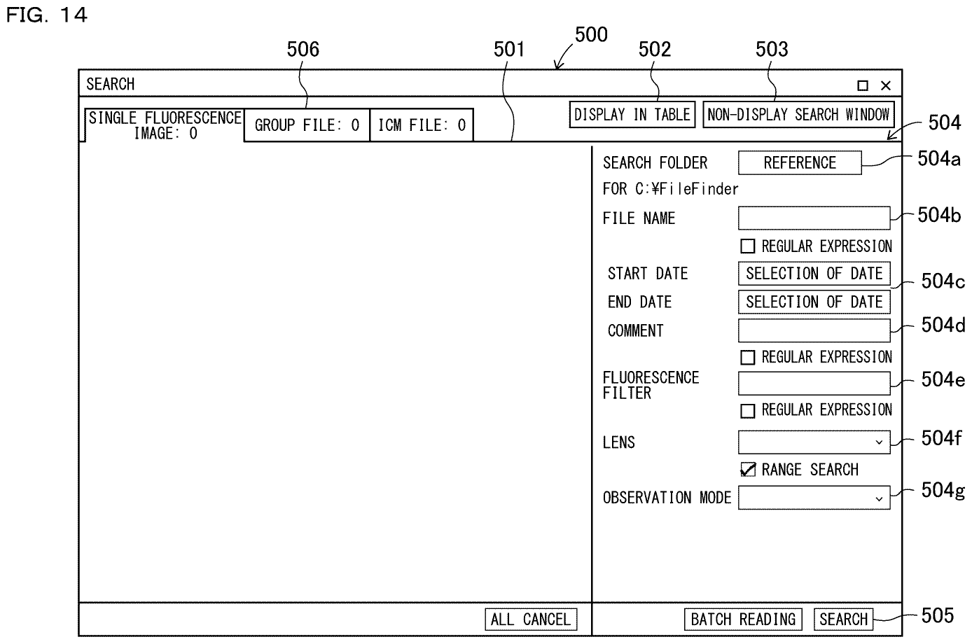

FIG. 14 illustrates a display screen 500 that the display control section 60 generates and causes the display section 21 to display. The display screen 500 is provided with a result display region 501 for displaying a search result, a result display switching button 502, a non-display button 503 for non-displaying a search condition setting region (search window) 504, and a search execution button 505. In a case where the non-display button 503 is not operated, the search condition setting region 504 is displayed on the display screen 500. The result display region 501 is provided with three tabs 506 for switching screens.

When starting the search application that executes the search function, for example, the user operates a start button or the like provided on another user interface screen (not illustrated), or the user selects a menu from a menu bar or the like provided on another user interface screen (not illustrated). Therefore, the search application that executes the search function is started.

When the search application that executes the search function is started, the display control section 60 causes the display section 21 to display a display screen 500 illustrated in FIG. 14. The search condition setting region 504 is provided with a folder input region 504a for inputting a search folder. A folder input in the folder input region 504a is set as a search range by the search setting section 61, and an image stored in the folder input in the folder input region 504a becomes a search target by the search section 62.

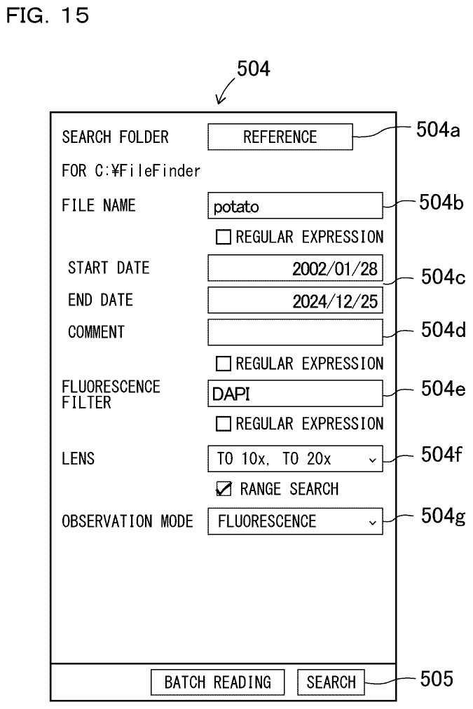

The search condition setting region 504 is provided with a file name input region 504b for inputting a file name as a search condition. FIG. 15 illustrates a state in which the file name is input, and the file name can be input in a text format as described above. The file name input to the file name input region 504b is set as a search condition by the search setting section 61, and the search section 62 searches for an image having the file name.

The search condition setting region 504 is provided with a date input region 504c for inputting an imaging date as a search condition. The period input in the date input region 504c is set as a search condition by the search setting section 61, and the search section 62 searches for an image that satisfies the condition of the period.

The search condition setting region 504 is provided with a comment input region 504d for inputting a comment as a search condition. The comment input to the comment input region 504d is set as a search condition by the search setting section 61, and the search section 62 searches for an image having the comment.

The search condition setting region 504 is provided with a filter information input region 504e for inputting fluorescence filter information used for imaging as a search condition. The fluorescence filter information input to the filter information input region 504e is set as a search condition by the search setting section 61, and the search section 62 searches for an image having the fluorescence filter information. As described above, the search setting section 61 can set the fluorescence filter information as the search condition, and specifically, the search setting section 61 receives the selection of the fluorescence filter and sets the received fluorescence filter as the search condition. The information processing method according to the present embodiment includes a step of setting fluorescence filter information as a search condition by the search setting section 61.

The search condition setting region 504 is provided with a lens information input region 504f for inputting lens information used for imaging as a search condition. The lens information input to the lens information input region 504f is set as a search condition by the search setting section 61, and the search section 62 searches for an image having the lens information.

The search condition setting region 504 is provided with an observation mode input region 504g for inputting an observation mode as a search condition. The observation mode input to the observation mode input region 504g is set as a search condition by the search setting section 61, and the search section 62 searches for an image captured in the observation mode.

The search condition may be input by the user, or the information may be automatically input as the search condition based on the metadata of the file. For example, the type of the fluorescence filter included in the metadata, the observation mode, and the like can be automatically input to the search condition, or the type of the fluorescence filter included in the metadata of the image, the observation mode, and the like can be automatically input to the search condition by dragging and dropping the image to the search condition setting region 504.

The notation variation of the search condition can be dealt with by using a regular expression. Furthermore, for example, a wildcard such as “*” or “?” can be used to deal with the notation variation. Moreover, it is also possible to deal with the notation variation by calculating the similarity using the editing distance or the like and considering that the similarity is greater than or equal to a certain value as matching.

After inputting a desired search condition in the search condition setting region 504, the user operates the search execution button 505 illustrated in FIG. 14. At the time of searching, the file reading section 68 reads a reference file. When detecting that the search execution button 505 is operated, the search section 62 searches for the single fluorescence image and the reference file that satisfy the search condition on the basis of the fluorescence filter information set by the search setting section 61 and the metadata corresponding to the single fluorescence image stored in the storage section 70 and the metadata corresponding to the reference file read by the file reading section 68. The information processing method according to the present embodiment includes a step of searching for the single fluorescence image and the reference file that satisfy the search condition in the search section 62 on the basis of the fluorescence filter information set as the search condition and the metadata corresponding to the single fluorescence image stored in the storage section 70 and the metadata corresponding to the reference file stored in the storage section 70.

When the search by the search section 62 is completed, the display control section 60 separates the single fluorescence image and the reference file searched by the search section 62 and displays a list thereof on the display section 21. This step is a display step of separating the searched single fluorescence image and the reference file and displaying a list thereof on the display section 21, and the information processing method according to the present embodiment also includes a display step.

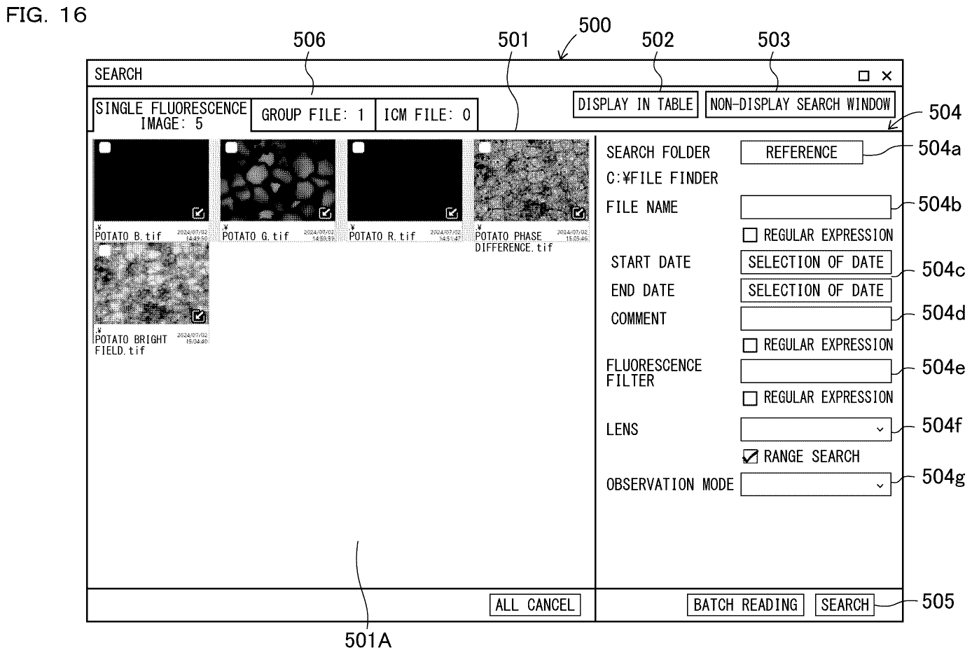

Specifically, the three tabs 506 of the result display region 501 include information indicating a single fluorescence image (single image), information indicating a reference file, and information indicating an ICM file, and each tab displays the number of searched images and files. As illustrated in FIG. 16, when the user operates the tab of the single image among the three tabs 506 of the result display region 501, the display control section 60 generates a first user interface screen 501A that displays a list of the single fluorescence images searched by the search section 62 and displays the generated screen on the display section 21.

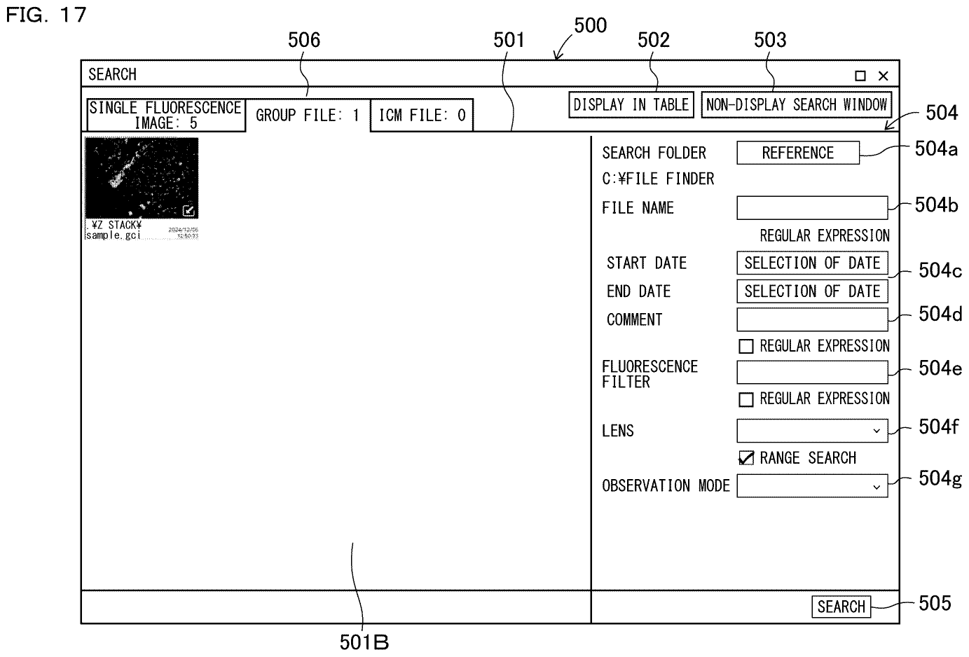

On the other hand, as illustrated in FIG. 17, when the user operates the tab of the group file among the three tabs 506 of the result display region 501, the display control section 60 generates a second user interface screen 501B displaying a list of the group files among the reference files searched by the search section 62, and displays the second user interface screen 501B on the display section 21. Furthermore, although not illustrated, when the user operates the tab of the ICM file among the three tabs 506 of the result display region 501, the display control section 60 generates a third user interface screen displaying a list of the ICM files among the reference files searched by the search section 62 and displays the third user interface screen on the display section 21.

In this manner, the display control section 60 causes the display section 21 to display the tab 506 for switching between the first user interface screen 501A and the second user interface screen 501B. The display control section 60 generates display data corresponding to one selected tab on the basis of one tab selected from the three tabs 506 indicating each of the single fluorescence image, the reference file, and the ICM file displayed on the display section 21. Therefore, the user can switch between the list display of the single fluorescence images and the list display of the reference files with a simple operation.

Note that, in addition to the tab 506, for example, an icon or a character string can be used as the information indicating each of the single fluorescence image and the reference file. The display position of the information indicating each of the single fluorescence image and the reference file is not limited to the upper portion, and may be anywhere on the screen.

A form other than the switching by the tab 506 can also be adopted. For example, the display control section 60 may cause the display section 21 to display the first user interface screen 501A and the second user interface screen 501B in different windows. In this case, both windows may be disposed such that the window of the first user interface screen 501A and the window of the second user interface screen 501B partially overlap each other, or both windows may be disposed side by side in the up and down direction or the left-right direction so as not to overlap each other at all.

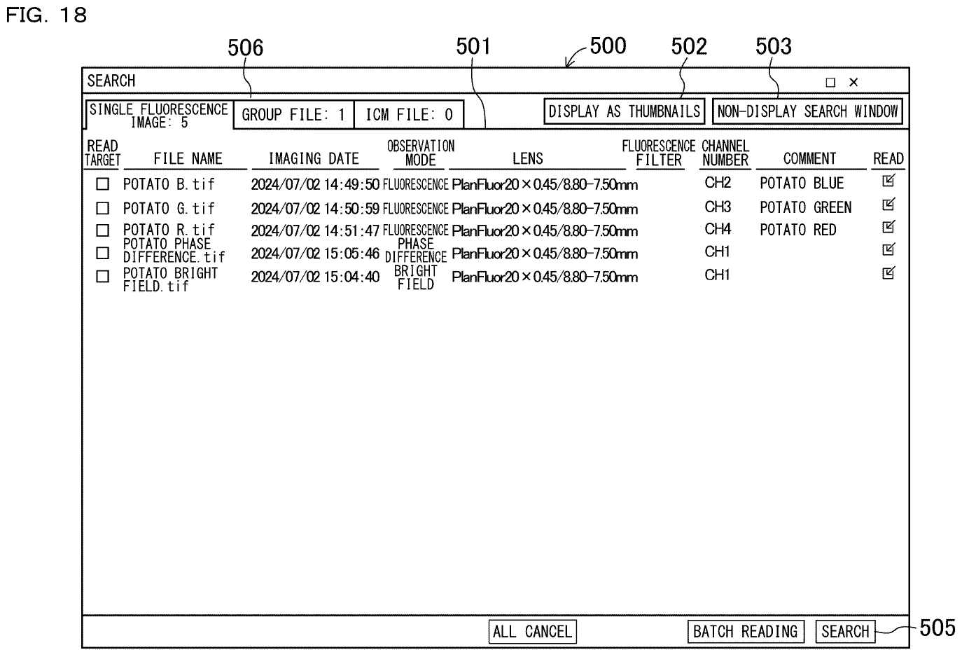

When the result display switching button 502 is operated by the user, the display control section 60 displays the metadata associated with the single fluorescence image in a table format as illustrated in FIG. 18. Therefore, the user can confirm the metadata.

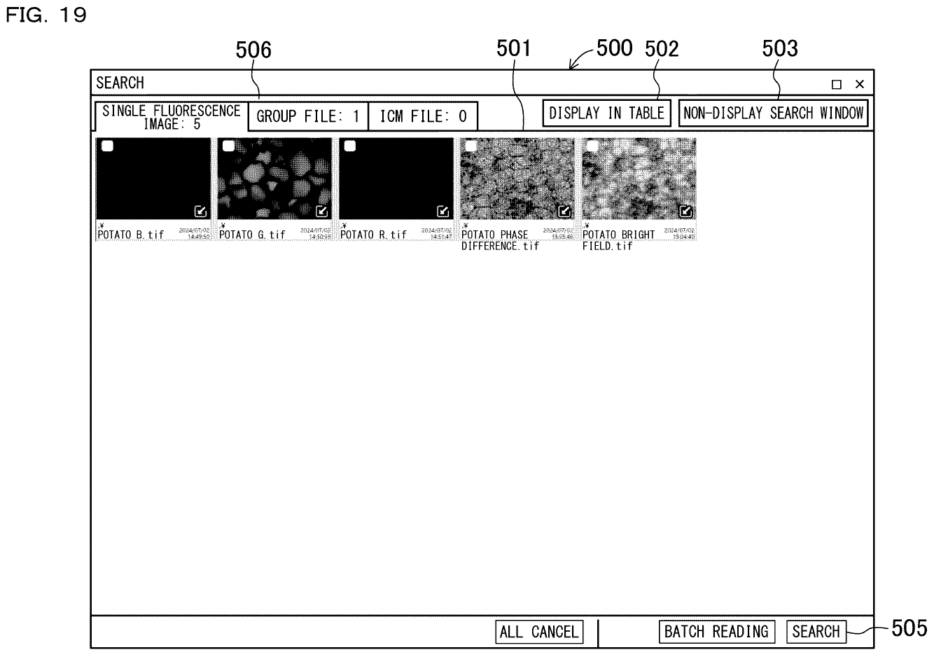

In FIG. 18, since the metadata is displayed in a table format, the result display switching button 502 is a thumbnail display button. When the user operates the thumbnail display button in FIG. 18, the display control section 60 displays the thumbnail image of the single fluorescence image as illustrated in FIG. 19. In this manner, the display of the metadata and the display of the thumbnail images can be switched as necessary.

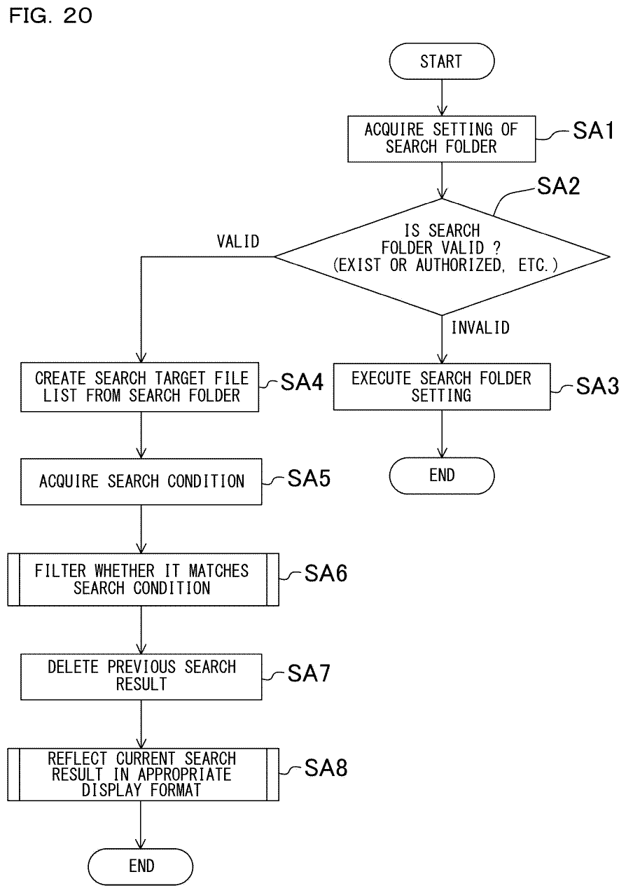

FIG. 20 is a flowchart illustrating an example of the search process, and details of the search process will be described on the basis of this flowchart. The process illustrated in the flowchart starts when the search execution button 505 illustrated in FIG. 14 is operated. In step SA1, the setting of the search folder is acquired. In step SA2, it is determined whether or not the search folder acquired in step SA1 is valid. In a case where the search folder is invalid, the process proceeds to step SA3 to execute the search folder setting.

In a case where the search folder is valid, the process proceeds to step SA4, and a search target file list is created from the search folder. In step SA5, the search condition of the search setting section 61 is acquired. In step SA6, the search section 62 searches the data as the search target, and performs filtering of the search target as to whether or not the data matches the search condition. In step SA7, in a case where there is a previous search result, the previous search result is deleted. In step SA8, the display control section 60 causes the display section 21 to display the current search result in an appropriate display format.

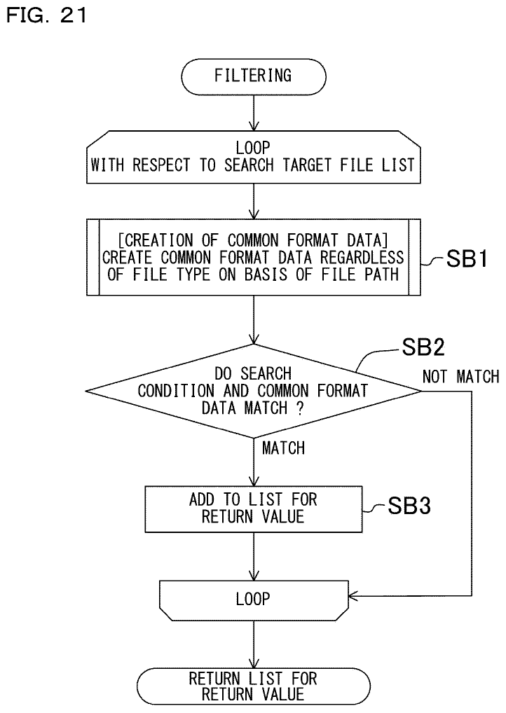

Details of the filtering processing of step SA6 are illustrated in the flowchart of FIG. 21. In the flowchart of FIG. 21, steps SB1 to SB3 are looped with respect to the search target file list. In step SB1, common format data is created. Specifically, the common format data regardless of the file type (single fluorescence image, group file and ICM file) is created on the basis of the file path. The common format data is search metadata used for searching a single fluorescence image, a group file, and an ICM file.

In step SB2, it is determined whether or not the search condition and the common format data match. In step SB1, since the common format data is created from different data configurations in the single fluorescence image, the group file, and the ICM file, it is possible to determine that files of different types match in the same portion. In a case where they do not match, the process proceeds to a loop. In a case where they match, the process proceeds to step SB3 to add them to the list for a return value.

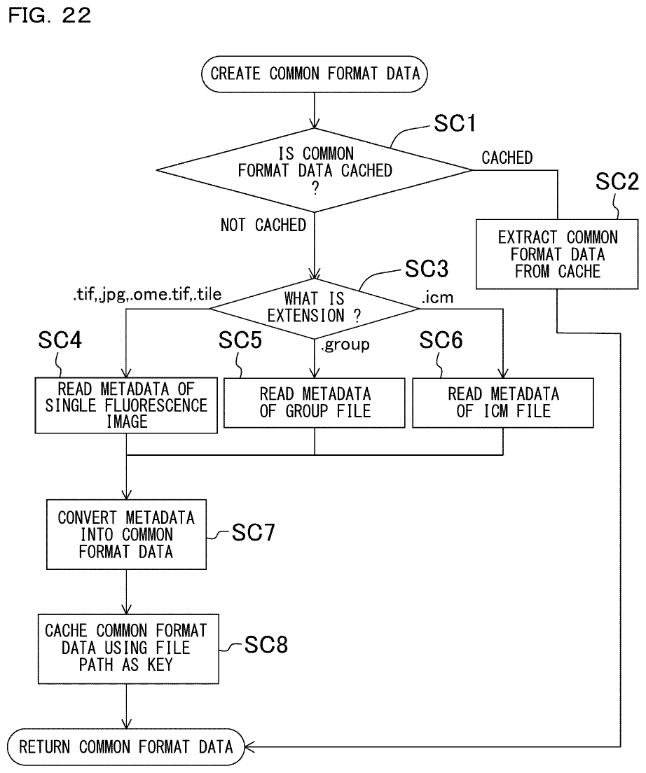

FIG. 22 is a flowchart illustrating details of a creation process of the common format data. In step SC1, it is determined whether or not the common format data is stored in a cache memory 69 (illustrated in FIG. 2) included in the electronic computer 2. In a case where the common format data is stored in the cache memory 69, the process proceeds to step SC2, and the common format data is extracted from the cache memory 69, and then the common format data is returned.

In a case where the common format data is not stored in the cache memory 69, the process proceeds to step SC3. In step SC3, an extension of the image is determined. When the extension of the image is any one of “.tif”, “.jpg”, “.ome.tif”, and “.tile”, the process proceeds to step SC4, and the metadata of the single fluorescence image is read. When the extension of the image is “group”, the process proceeds to step SC5, and the metadata of the group file is read. When the extension of the image is “.icm”, the process proceeds to step SC6, and the metadata of the ICM file is read.

In step SC7, the metadata read in step SC4 to 6 is converted into the common format data. In step SC8, the common format data is stored in the cache memory 69 using the file path as a key.

When the metadata is converted into the common format data, different data configurations in the single fluorescence image, the group file, and the ICM file are converted into the common format data using not only the common portion but also the unique portion.

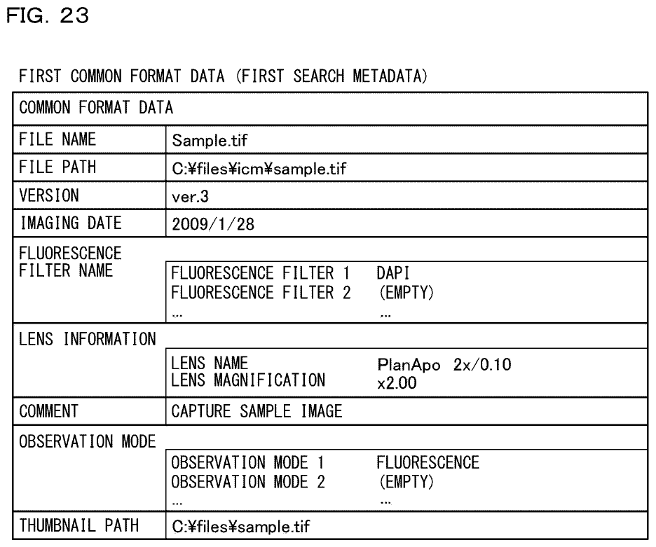

The creation section 63 creates first common format data (first search metadata) having a plurality of cells as illustrated in FIG. 23 on the basis of the metadata corresponding to the single fluorescence image. The file name of the metadata of the single fluorescence image is input to the cell having the file name of the first common format data. The file path of the metadata of the single fluorescence image is input to the cell of the file path of the first common format data. In the cell of the version of the first common format data, the version of the metadata of the single fluorescence image is input. In the cell of the imaging date of the first common format data, the imaging date of the image information of the metadata of the single fluorescence image is input. The fluorescence filter name of the imaging condition of the metadata of the single fluorescence image is input to the cell of the fluorescence filter name of the first common format data. The lens information of the metadata of the single fluorescence image is input to the cell of the lens information of the first common format data. The comment of the image information of the metadata of the single fluorescence image is input to the cell of the comment of the first common format data. An observation mode of an imaging condition of the metadata of the single fluorescence image is input to the cell of the observation mode of the first common format data. The file path of the metadata of the single fluorescence image is input to the cell of the thumbnail path of the first common format data.

As described above, the first common format data includes the plurality of cells to which the imaging condition is input. Then, the creation section 63 acquires a single imaging condition from the metadata corresponding to the single fluorescence image, and allocates the acquired imaging condition to one cell among the plurality of cells to which the imaging condition included in the first common format data is input.

The creation section 63 stores the file path of the single fluorescence image and the first common format data in the cache memory 69 (illustrated in FIG. 2). When creating the first common format data, the creation section 63 determines whether or not the first common format data exists in the cache memory 69 on the basis of the file path of the single fluorescence image (step SC1 in FIG. 22).

In a case where it is determined that the first common format data exists in the cache memory 69, the creation section 63 acquires the first common format data from the cache memory 69, and in a case where it is determined that the first common format data does not exist in the cache memory 69, the creation section creates the first common format data on the basis of the metadata corresponding to the single fluorescence image.

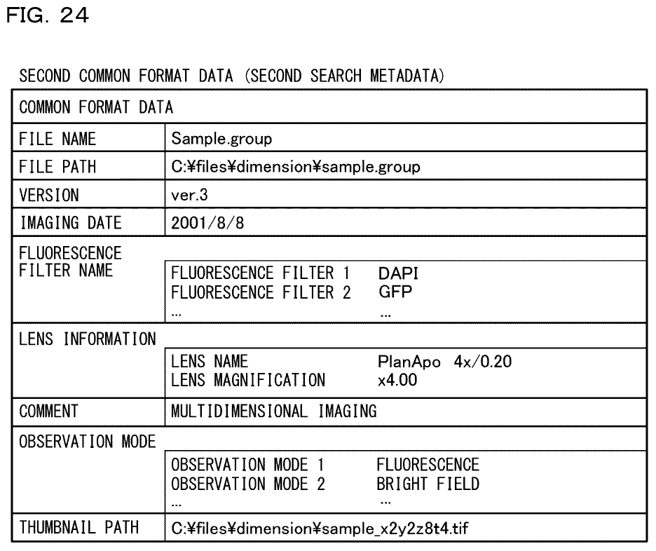

The creation section 63 creates second common format data (second search metadata) having a plurality of cells as illustrated in FIG. 24 on the basis of the metadata corresponding to the group file. In the cell of the file name of the second common format data, the file name of the metadata of the group file is input. The file path of the metadata of the group file is input to the cell of the file path of the second common format data. The version of the metadata of the group file is input to the cell of the version of the second common format data. In the cell of the imaging date of the second common format data, the imaging date of the image information of the metadata of the group file is input. The fluorescence filter names of the imaging conditions in the metadata corresponding to each single fluorescence image, with duplicates removed, are input to the cells of the fluorescence filter names in the second common format data. The lens information of the metadata of the group file is input to the cell of the lens information of the second common format data. In the cell of the comment of the second common format data, the comment of the image information of the metadata of the group file is input. The observation modes of the imaging conditions in the metadata corresponding to each single fluorescence image, with duplications removed, are input to the cells of the observation modes in the second common format data. In the cell of the thumbnail path of the second common format data, the center of each dimension is calculated from the imaging method, the image stitching setting, the Z stack setting, and the time lapse setting, and the file path of the metadata corresponding to the central image file of each dimension is input.

As described above, the second common format data also includes the plurality of cells to which the imaging condition is input. Then, the creation section 63 acquires a plurality of imaging conditions from the metadata corresponding to the reference file, and sequentially allocates the acquired plurality of imaging conditions to each of the plurality of cells to which the imaging conditions included in the second common format data are input.

The creation section 63 stores the file path of the reference file and the second common format data in the cache memory 69 included in the electronic computer 2. When creating the second common format data, the creation section 63 determines whether or not the second common format data exists in the cache memory 69 on the basis of the file path of the reference file. In a case where it is determined that the second common format data exists in the cache memory 69, the creation section 63 acquires the second common format data from the cache memory 69, and in a case where it is determined that the second common format data does not exist in the cache memory 69, the creation section creates the second common format data on the basis of the metadata corresponding to the reference file.

As described above, since the file path is stored as a key in the cache memory 69 in association with the common format data, for example, in a case where the search is repeatedly executed, the common format data can be obtained at a high speed by reading the common format data from the cache memory 69 in the second and subsequent searches. Furthermore, each file may be stored in an external medium (storage section 70), and in such a case, it is difficult to create a database. However, by using a file path that can ensure uniqueness as a key, it is possible to realize high-speed processing.

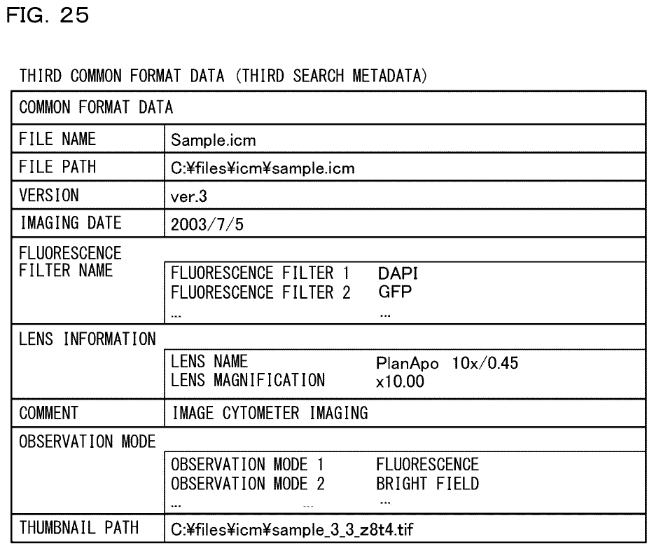

The creation section 63 creates third common format data (third search metadata) having a plurality of cells as illustrated in FIG. 25 on the basis of the metadata corresponding to the ICM file. In the cell of the file name of the third common format data, the file name of the metadata of the ICM file is input. The file path of the metadata of the ICM file is input to the cell of the file path of the third common format data. In the cell of the version of the third common format data, the version of the metadata of the ICM file is input. In the cell of the imaging date of the third common format data, the imaging date of the image information of the metadata of the ICM file is input. The fluorescence filter names in the list of the imaging conditions in the metadata corresponding to each group file, with duplicates removed, are input to the cells of the fluorescence filter names in the third common format data. The lens information of the metadata of the ICM file is input to the cell of the lens information of the third common format data. In the cell of the comment of the third common format data, the comment of the image information of the metadata of the ICM file is input. The observation modes in the list of the imaging conditions in the metadata corresponding to each group file, with duplicates removed, are input to the cells of the observation mode in the third common format data. In the cell of the thumbnail path of the third common format data, the center of each dimension is calculated from the imaging method, the image stitching setting, the Z stack setting, and the time lapse setting, and the file path of the metadata corresponding to the central image file of each dimension is input.

As described above, the third common format data also includes the plurality of cells to which the imaging condition is input. Then, the creation section 63 acquires a plurality of imaging conditions from the metadata corresponding to the ICM file, and sequentially allocates the acquired plurality of imaging conditions to each of the plurality of cells to which the imaging conditions included in the third common format data are input.

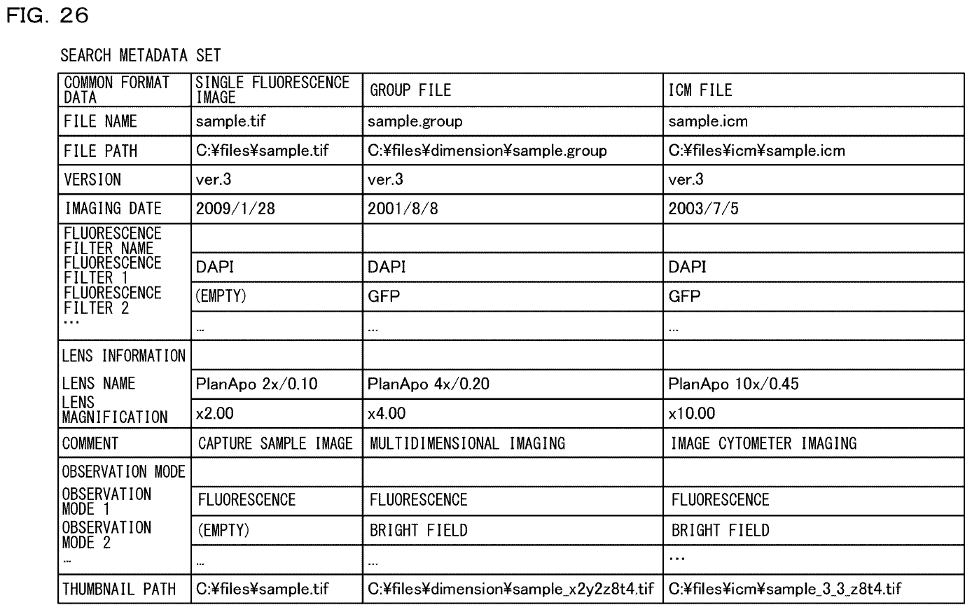

The creation section 63 creates a common format data set (search metadata set) as illustrated in FIG. 26. That is, the creation section 63 acquires the first common format data, the second common format data, and the third common format data, and creates the common format data set including the first common format data, the second common format data, and the third common format data on the basis of the first common format data, the second common format data, and the third common format data. The creation section 63 may create a common format data set including the first common format data and the second common format data on the basis of the first common format data and the second common format data.

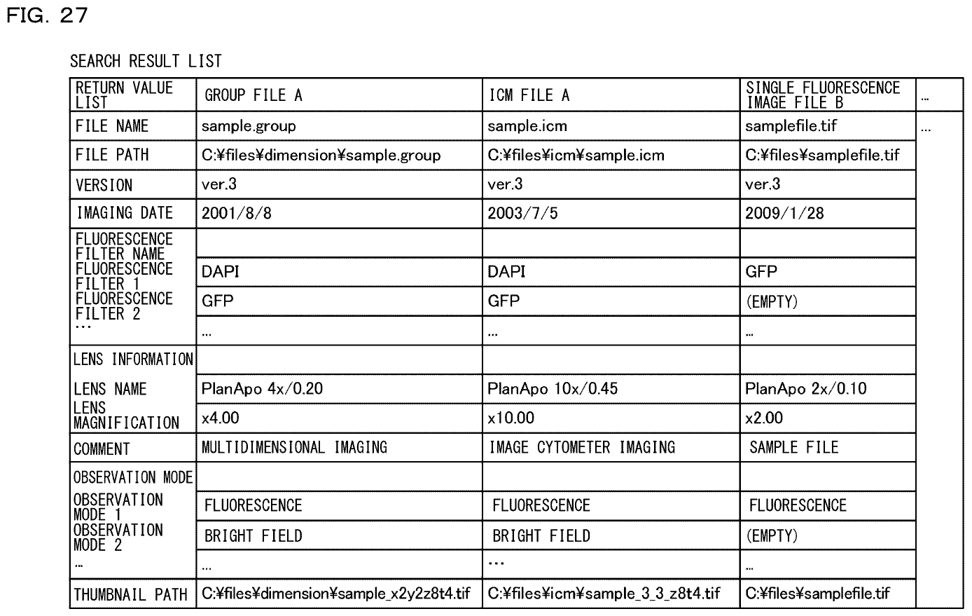

In step SB2 illustrated in FIG. 21, the search section 62 acquires the common format data set (illustrated in FIG. 26) created by the creation section 63 and the fluorescence filter information set by the search setting section 61. Then, the search section 62 searches the metadata satisfying the search condition based on the acquired common format data set and the fluorescence filter information, and generates a search result list obtained by extracting the metadata satisfying the search condition from the common format data set. FIG. 27 illustrates an example of the search result list generated by the search section 62. In this way, the search results of the group file, the ICM file, and the single fluorescence image file are obtained in the form of a list.

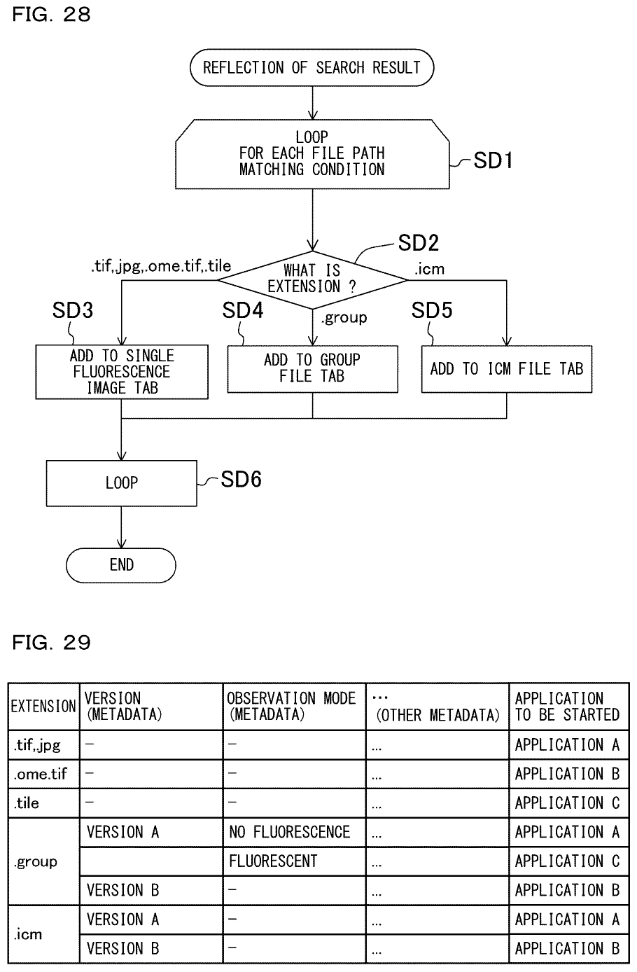

Next, details of the display control section 60 will be described. FIG. 28 is a flowchart illustrating details of a display process by the display control section 60. The process illustrated in this flowchart is started at the timing when the search result list is generated or at any subsequent timing.

In step SD1, loop processing of executing steps SD2 to SD5 for each file path matching the search condition is started. In step SD2, an extension is determined. When the extension of the file is any one of “.tif”, “.jpg”, “.ome.tif”, and “.tile”, the process proceeds to step SD3, and the file is added to the tab 506 (illustrated in FIG. 14) of the single fluorescence image. When the extension of the file is “group”, the process proceeds to step SD4, and the file is added to the tab 506 of the group file. When the extension of the file is “.icm”, the process proceeds to step SD5, and the file is added to tab 506 of the ICM file. As described above, since the file type can be determined by the extension, each file can be classified so as to be displayed at an appropriate location.

This classification processing is executed by the file attribute classification section 64 illustrated in FIG. 2. The file attribute classification section 64 classifies the single fluorescence image and the reference file searched by the search section 62 on the basis of the extension which is one of the file attributes corresponding to the single fluorescence image stored in the storage section 70 and the file attribute corresponding to the reference file.

Note that the classification of the file is not limited to the classification on the basis of the extension of the file, and the classification may be performed on the basis of the correctly read type by testing whether or not each of the plurality of images can be correctly read. Furthermore, a common region indicating a file type may be prepared in the metadata, and the common region may be read and classified.

The display control section 60 displays the single fluorescence image having the file attribute classified into the single fluorescence image by the file attribute classification section 64 on the first user interface screen 501A as illustrated in FIG. 16, for example. Furthermore, the display control section 60 displays the reference file having the file attribute classified into the reference file by the file attribute classification section 64 on the second user interface screen 501B as illustrated in FIG. 17, for example.

In step SD6 of FIG. 28, the loop processing is continued, and when the loop processing is completed, this flow ends.

By executing the processing illustrated in FIG. 28, the display control section 60 can display the single fluorescence image corresponding to the metadata on the display section 21 in a case where the metadata corresponding to the single fluorescence image is included in the search result list, and can display each of the plurality of single fluorescence images bundled by the reference file corresponding to the metadata on the display section 21 in a case where the metadata corresponding to the reference file is included in the search result list.

When the display control section 60 specifies the single fluorescence image to be displayed on the display section 21, it is also possible to specify the single fluorescence image to be displayed on the display section 21 on the basis of the focus value included in the metadata of each of the plurality of single fluorescence images bundled by the reference file. That is, since the focus value is included in the metadata of each image, it is possible to specify the most focused image by acquiring the focus values of the plurality of images. Then, by displaying the most focused image on the display section 21, the user can easily observe the image.

Next, start-up of an analysis application for analyzing an observation image will be described. For example, in a case where the single fluorescence image illustrated in FIG. 16 is displayed in a list, the user can perform a selection operation of a predetermined single fluorescence image. When the user performs a selection operation of a predetermined single fluorescence image, the reception section 65 receives the selection of the predetermined single fluorescence image.

Furthermore, in a case where a list of reference files is displayed, the user can perform a predetermined reference file selection operation. When the user performs an operation of selecting a predetermined reference file, the reception section 65 receives the selection of the predetermined reference file.

Moreover, in either case of the display in the table format illustrated in FIG. 18 or the display in the thumbnail format illustrated in FIG. 19, selection of a single fluorescence image and selection of a reference file are possible, and also in these cases, the reception section 65 receives selection of a predetermined single fluorescence image and selection of a predetermined reference file.

The determination section 66 determines an analysis application to be started on the basis of metadata corresponding to a predetermined single fluorescence image or metadata corresponding to a predetermined reference file received by the reception section 65. At the time of determination, for example, an analysis application determination table (illustrated in FIG. 29) stored and held in the storage section 70 or the like is used. An extension of each image, a version included in metadata of each image, an observation mode included in metadata of each image, other metadata corresponding to each image, and an analysis application to be started are arranged in a horizontal direction (row arrangement direction) of the analysis application determination table. As described above, the analysis application determination table has a form in which the analysis application to be started is associated with each of the metadata corresponding to the single fluorescence image and the metadata corresponding to the reference file

The determination section 66 acquires the extension, version, observation mode, and other metadata of each image, determines a combination thereof, and determines whether to start the analysis application A, start the analysis application B, or start the analysis application C on the basis of the determination result. That is, the determination section 66 determines the analysis application to be started on the basis of the metadata corresponding to the predetermined single fluorescence image or the metadata corresponding to the predetermined reference file received by the reception section 65 and the analysis application determination table. Note that the analysis applications A to C are examples, and the number of analysis applications is not limited to three.

When the analysis application corresponding to the single fluorescence image is determined by the determination section 66, the start-up section 67 starts the determined analysis application. Furthermore, when the determination section 66 determines the analysis application corresponding to the reference file, the start-up section 67 starts the determined analysis application. It is also possible to connect to an application or software other than the analysis application described above, and in this case, it is sufficient that a command line argument can be designated.

As described above, in the present embodiment, the information processing method can be executed by using the fluorescence microscope A. An information processing method includes the steps of: causing a storage section to store a plurality of single fluorescence images acquired by different fluorescence filters, each of the single fluorescence images including metadata, and causing the storage section to store a reference file that bundles some single fluorescence images from among the plurality of single fluorescence images; setting fluorescence filter information as a search condition; searching for the single fluorescence images and the reference file that satisfy the search condition on the basis of the fluorescence filter information set as the search condition, and metadata corresponding to the stored single fluorescence images and metadata corresponding to the stored reference file, and separating the searched single fluorescence images and reference file and causing a display section 21 to display a list of the separated single fluorescence images and reference file. A program for causing the electronic computer 2 to execute this information processing method is also included in the present embodiment. The program can be recorded on a recording medium such as an optical disk or a magnetic recording medium. In this case, a recording medium on which the program is recorded is also included in the present embodiment. Furthermore, the program may be stored in a server, downloaded by the user using a communication network such as the Internet, and installed in the electronic computer 2. Furthermore, the use of the program is not limited to the use in the electronic computer 2, and the program may be stored in a server and used by the user accessing the server via a communication network such as the Internet. That is, the program is not limited to being executed by the electronic computer 2, and may be executed by an external server or the like.

The above-described embodiments are merely examples in all respects, and should not be construed in a limiting manner. Moreover, all modifications and changes falling within the equivalent scope of the claims are within the scope of the present invention.

As described above, the fluorescence microscope according to the present disclosure can be used, for example, in the case of observing a biological specimen or the like.

What is claimed is:

1. An information processing apparatus configured to search for a fluorescence image of an observation target, the information processing apparatus comprising:
a storage section configured to store a plurality of single fluorescence images acquired by different fluorescence filters, each of the single fluorescence images including metadata, and store a reference file that bundles some single fluorescence images from among the plurality of single fluorescence images;
a search setting section configured to set fluorescence filter information as a search condition;
a search section configured to search for the single fluorescence images and the reference file that satisfy the search condition on a basis of the fluorescence filter information set by the search setting section, and metadata corresponding to the single fluorescence images and metadata corresponding to the reference file stored in the storage section; and
a display control section configured to separate the single fluorescence images and the reference file searched by the search section and cause a display section to display a list of the separated single fluorescence images and reference file.

2. The information processing apparatus according to claim 1, wherein the display control section causes the display section to display a first user interface screen that displays a list of the single fluorescence images searched by the search section and a second user interface screen that displays a list of the reference file searched by the search section.

3. The information processing apparatus according to claim 2, wherein the display control section causes the display section to display a tab for switching between the first user interface screen and the second user interface screen.

4. The information processing apparatus according to claim 2, wherein the display control section causes the display section to display the first user interface screen and the second user interface screen in different windows.

5. The information processing apparatus according to claim 1, wherein
the metadata corresponding to the single fluorescence images includes a single imaging condition, and
the metadata corresponding to the reference file includes a plurality of imaging conditions based on an imaging condition included in each of the single fluorescence images bundled by the reference file.

6. The information processing apparatus according to claim 5, further comprising a creation section configured to create first search metadata on a basis of the metadata corresponding to the single fluorescence images, create second search metadata on a basis of the metadata corresponding to the reference file, and create a search metadata set on a basis of the first search metadata and the second search metadata.

7. The information processing apparatus according to claim 6, wherein
the first search metadata and the second search metadata include a plurality of cells into which an imaging condition is input, and
the creation section acquires a single imaging condition from the metadata corresponding to the single fluorescence images, allocates the acquired imaging condition to one cell among the plurality of cells into which an imaging condition included in the first search metadata is input, acquires a plurality of imaging conditions from the metadata corresponding to the reference file, and sequentially allocates the acquired plurality of imaging conditions to each of the plurality of cells into which an imaging condition included in the second search metadata is input.

8. The information processing apparatus according to claim 6, wherein the creation section stores a file path of the single fluorescence images and the first search metadata in a cache memory, and stores a file path of the reference file and the second search metadata in a cache memory.

9. The information processing apparatus according to claim 8, wherein
the creation section
determines whether or not the first search metadata exists in the cache memory on a basis of the file path of the single fluorescence images, acquires the first search metadata from the cache memory in a case where it is determined that the first search metadata exists in the cache memory, and creates the first search metadata on a basis of the metadata corresponding to the single fluorescence images in a case where it is determined that the first search metadata does not exist in the cache memory, and
determines whether or not the second search metadata exists in the cache memory on a basis of the file path of the reference file, acquires the second search metadata from the cache memory in a case where it is determined that the second search metadata exists in the cache memory, and creates the second search metadata on a basis of the metadata corresponding to the reference file in a case where it is determined that the second search metadata does not exist in the cache memory.

10. The information processing apparatus according to claim 6, wherein
the search section searches metadata satisfying a search condition on a basis of the fluorescence filter information set by the search setting section and the search metadata set created by the creation section, and generates a search result list obtained by extracting metadata satisfying the search condition from the search metadata set, and
the display control section causes, in a case where the search result list includes metadata corresponding to single fluorescence images, the display section to display the single fluorescence images corresponding to the metadata, and causes, in a case where the search result list includes metadata corresponding to a reference file, the display section to display each of a plurality of single fluorescence images bundled by the reference file corresponding to the metadata.

11. The information processing apparatus according to claim 10, wherein the display control section specifies a single fluorescence image to be displayed on the display section on a basis of a focus value included in metadata of each of the plurality of single fluorescence images bundled by the reference file.

12. The information processing apparatus according to claim 2, further comprising a file attribute classification section configured to classify the single fluorescence images and the reference file searched by the search section on a basis of a file attribute corresponding to the single fluorescence images and a file attribute corresponding to the reference file stored in the storage section,
wherein the display control section displays, on the first user interface screen, the single fluorescence images having the file attribute classified as the single fluorescence images by the file attribute classification section, and displays, on the second user interface screen, the reference file having the file attribute classified as the reference file by the file attribute classification section.

13. The information processing apparatus according to claim 12, wherein the file attribute classification section classifies the single fluorescence images and the reference file on a basis of an extension as the file attribute and the file attribute corresponding to the reference file.

14. The information processing apparatus according to claim 1, wherein the display control section causes the display section to display information indicating each of the single fluorescence images and the reference file, and generates display data corresponding to one piece of information selected on a basis of one piece of information selected from information indicating each of the single fluorescence images and the reference file displayed on the display section.

15. The information processing apparatus according to claim 1, wherein
the information processing apparatus holds an application determination table in which an application to be started is associated with each of the metadata corresponding to the single fluorescence images and the metadata corresponding to the reference file,
the information processing apparatus further comprising:
a reception section configured to receive selection of a predetermined single fluorescence image or a predetermined reference file from the single fluorescence images or the reference file whose list is displayed on the display section by the display control section;
a determination section configured to determine an application to be started on a basis of metadata corresponding to the predetermined single fluorescence image or metadata corresponding to the predetermined reference file received by the reception section, and the application determination table; and
a start-up section configured to start the application determined by the determination section.

16. The information processing apparatus according to claim 1, wherein
the reference file includes a first reference file that bundles a plurality of single fluorescence images acquired at different positions, and a second reference file that bundles a plurality of single fluorescence images, and
the first reference file bundles the second reference file that bundles a plurality of single fluorescence images acquired at each position.

17. The information processing apparatus according to claim 1, wherein the search setting section receives selection of the fluorescence filters and sets the received fluorescence filters as a search condition.

18. The information processing apparatus according to claim 1, further comprising a file reading section configured to read the reference file,
wherein the search section searches for the single fluorescence images and the reference file that satisfy the search condition on a basis of the fluorescence filter information set by the search setting section, the metadata corresponding to the single fluorescence images stored in the storage section, and metadata corresponding to the reference file read by the file reading section.

19. An information processing method of searching for a fluorescence image of an observation target, the information processing method comprising the steps of:
causing a storage section to store a plurality of single fluorescence images acquired by different fluorescence filters, each of the single fluorescence images including metadata, and causing the storage section to store a reference file that bundles some single fluorescence images from among the plurality of single fluorescence images;
setting fluorescence filter information as a search condition in a search setting section;
searching for the single fluorescence images and the reference file that satisfy the search condition in a search section on a basis of the fluorescence filter information set as the search condition, and metadata corresponding to the stored single fluorescence images and metadata corresponding to the stored reference file, and
separating the searched single fluorescence images and reference file and causing a display section to display a list of the separated single fluorescence images and reference file.

20. A program storage medium storing a program for causing a computer to execute the steps of:
storing a plurality of single fluorescence images acquired by different fluorescence filters, each of the single fluorescence images including metadata, and storing a reference file that bundles some single fluorescence images from among the plurality of single fluorescence images;
setting fluorescence filter information as a search condition;
searching for the single fluorescence images and the reference file that satisfy the search condition on a basis of the fluorescence filter information set as the search condition, and metadata corresponding to the stored single fluorescence images and metadata corresponding to the stored reference file; and
separating the searched single fluorescence images and reference file and causing a display section to display a list of the separated single fluorescence images and reference file.
